(19) United States
(12) Patent Application Publication
(10) Pub. No.: US 2026/0232355 A1
(43) Pub. Date: Aug. 13, 2026

(54) APPARATUS FOR ADVANCING SURGICAL GUIDE WIRE ENGAGEMENT DEVICE

(71) Applicant: Axia Orthopedics, Inc., Portland, OR (US)

(72) Inventors: Andrew William Seykora, Portland, OR (US); Casey M. Chambers, Portland, OR (US); James Michelinie, Portland, OR (US); Rebecca Schaldach, Portland, OR (US)

(21) Appl. No.: 19/051,920

(22) Filed: Feb. 12, 2025

(51) Int. Cl. A61B 17/68

(52) U.S. Cl. CPC A61B 17/68; A61B 2017/681

(57) ABSTRACT

Medical devices are disclosed for compression of tissue and/or implants via a guide wire during provisional reduction in fracture fixation surgeries and other types of surgeries. Accordingly, an example hand-held medical device advancement apparatus includes a housing defining a handle and a body intersecting the handle. The apparatus may also include a mechanically-actuated mechanism that is actuatable to advance a medical device located in front of the body along a guide wire. The apparatus may further include a nose portion that extends distally away from the body and that retracts toward the body against spring bias when a trigger on the apparatus is actuated to concurrently advance the body and the medical device along the wire while the medical device is obstructed by a surface in front of the medical device. Methods for manufacturing, providing, and using the medical device(s) and/or apparatus(es) are also disclosed.

### FIELD

The disclosure below relates generally to apparatuses for advancing surgical guide wire engagement devices along surgical guide wires during provisional fracture reductions and other orthopedic surgical procedures.

### BACKGROUND

Kirschner wires (k-wires) are often used to aid in the provisional alignment and reduction of fractures and implants during fracture fixation surgery in a human patient. K-wires come in different diameters, lengths, and materials. They typically have at least one trochanteric drilling tip, and in some cases a fluted tip. Some are smooth along the length and others have a threaded tip or ridges at the tip to improve bone purchase. K-wires help in orthopedic surgery as they create a very small hole that minimizes impact on the bone, thus allowing a surgeon several attempts at provisional reduction placement without significant bone loss. K-wires typically align bone fragments and implants in two planes of fixation.

However, one downside of k-wires is that they offer little to no compression (e.g., they do not align or compress in the third plane). Olive wires (OWs) and plate tacks (PTs) have been used to offer a slight improvement in reduction. Yet even here, the “olive” or “tack” is in a fixed position which prevents ideal wire purchase and/or depth. For threaded OWs and PTs, the bone thread interface often gets stripped because the wire is inserted at high speeds with a drill and advanced until the olive or tack is stopped by an implant or tissue, thus stripping the bone thread interface because the wire is still spinning but no longer advancing. One additional problem with OWs and PTs is that when inserted in a screw hole of a plate, a starting position off center can result in the olive or tack “kicking” the plate to the side as the olive or tack interacts with the plate hole.

There are also issues with locating and configuring a clamp to assist with provisional reduction, as this often requires another incision that is otherwise unnecessary. Also, the clamp might have to clamp down on other important and healthy bone, vascular structure, and nerve structure, which can damage those parts of the body. Clamps are also often quite crude in terms of the pressure they apply.

There are currently no adequate solutions to the foregoing problems.

### SUMMARY

Accordingly, the disclosure below relates to technology that allows precise alignment and compression of tissue and/or implants over a k-wire, Steinmann pin, and/or other alignment mechanism during provisional reduction of fractures and implants during fracture fixation surgery in a human patient. A surgeon or other physician may thus verify an intended alignment, minimizing impact on the bone during provisional reduction placement without significant bone loss and prior to permanent reduction placement/fixation. Thus, certain example devices discussed below may establish a movable pill or tack that can lock at any point along the k-wire, Steinmann pin, or other alignment wire/element. Additionally, example advancement apparatuses discussed below may be used to advance the pill or track down the wire in a controlled fashion that avoids unnecessary bone loss.

Therefore, in one aspect a hand-held medical device advancement apparatus includes a housing defining a handle and an elongated body intersecting the handle. The apparatus also includes a mechanically-actuated mechanism coupled to the housing. The mechanically-actuated mechanism includes a trigger that is actuatable to advance a medical device located in front of the elongated body along a surgical guide wire while the surgical guide wire concurrently extends through both the medical device and the elongated body. The apparatus further includes a nose portion that extends distally away from the elongated body and that retracts toward the elongated body against spring bias when the trigger is actuated to concurrently advance the elongated body and the medical device along the surgical guide wire while the medical device is obstructed by a surface in front of the medical device.

In various instances, the surface may include a bone and/or a surgical plate.

Additionally, in some example embodiments the apparatus may include a compression spring that provides the spring bias. In one particular instance, the compression spring may have a maximum compression force amount below 150 N for reasons set forth below.

Also in non-limiting example embodiments, the nose portion may be lockable in a locked configuration in which the nose portion remains immobile with respect to the elongated body. In one particular example, the nose portion may include one or more tabs that are extendable into one or more respective cavities in the elongated body to lock the nose portion in the locked configuration.

Also, if desired the apparatus may include the medical device itself. In some instances, the apparatus and medical device may even be provided together as a kit that also includes other surgical components, such as the surgical guide wire.

Still further, in some example implementations, the mechanically-actuated mechanism may further include a bar and a cam plate. The cam plate may include an opening through which the surgical guide wire is extendable while also concurrently extending through the elongated body. The trigger may be coupled to the bar at a first end portion of the bar, and the bar may include a second end portion that engages the cam plate to move the cam plate based on actuation of the trigger. In one specific non-limiting example, the mechanically-actuated mechanism may be configured, via actuation of the trigger, to move the cam plate to cinch the surgical guide wire as extending through the opening and to advance the hand-held medical device advancement apparatus along the surgical guide wire based on additional movement of the cam plate under control of the trigger while the wire continues to be cinched.

In another aspect, an apparatus includes a housing defining a handle and a body intersecting the handle. The apparatus also includes a mechanism coupled to the housing. The mechanism includes an arm that is actuatable to advance a medical device located in front of the body along a surgical guide wire while the surgical guide wire concurrently extends through both the medical device and the body. The mechanism further includes a bar and a plate. The plate includes an opening through which the surgical guide wire is extendable while also concurrently extending through the body. The arm is coupled to the bar at a first end portion of the bar, and the bar includes a second end portion that engages the plate to move the plate based on actuation of the arm.

In various examples, the bar may move substantially parallel to a longitudinal axis of the body.

Also in some examples, the apparatus may include a nose portion that extends distally away from the body. The nose portion may also retract toward the body against spring bias when the arm is actuated to concurrently advance the body and the medical device along the surgical guide wire while the medical device is obstructed by a surface in front of the medical device. In some instances, the nose portion may even be lockable in a locked configuration in which the nose portion remains immobile with respect to the body. In one particular non-limiting embodiment, the apparatus may include one or more tabs that are extendable into one or more respective cavities in the apparatus to lock the nose portion in the locked configuration. Still further, the apparatus may include a compression spring that provides the spring bias. In non-limiting instances, the compression spring may have a maximum compression force amount below 150 N.

In addition, in some implementations the mechanism may be configured, via actuation of the arm, to move the plate to cinch the surgical guide wire and to move the apparatus along the surgical guide wire.

In still another aspect, an apparatus includes a housing defining a handle and a body coupled to the handle. The apparatus also includes a nose portion extending distally away from the body. The nose portion is retractable toward the body against spring bias when the apparatus is actuated to concurrently move the body and a medical device in front of the nose portion along a surgical guide wire while the medical device is obstructed by a surface in front of the medical device.

In some example embodiments, the apparatus may include an arm that is actuatable to concurrently move the body and the medical device forward along the surgical guide wire.

Also in some non-limiting embodiments, the nose portion may be lockable in a locked configuration in which the nose portion remains immobile with respect to the body.

The details of the present application, both as to its structure and operation, can best be understood in reference to the accompanying drawings, in which like reference numerals refer to like parts, and in which:

### BRIEF DESCRIPTION OF THE DRAWINGS

FIG. 1A shows a front isometric view of a first example embodiment of a martini medical device consistent with present principles;

FIG. 1B shows a rear isometric view of the first example embodiment of the martini medical device consistent with present principles;

FIGS. 1C-1G show various orthogonal views of the first example embodiment of the martini medical device consistent with present principles;

FIG. 2 shows an exploded view of the first example embodiment of the martini medical device consistent with present principles;

FIG. 3 shows a side cross-sectional view of the first example embodiment of the martini medical device consistent with present principles;

FIG. 4 shows a rear isometric view, with the cap removed, of the first example embodiment of the martini medical device consistent with present principles;

FIGS. 5A and 5B show orthogonal views of the first example embodiment of the martini medical device consistent with present principles to demonstrate different example force gauges that may be used;

FIG. 6A shows a side isometric view of a first example embodiment of a hand-held advancement apparatus that may be used to advance a martini medical device along a surgical guide wire consistent with present principles;

FIG. 6B shows an exploded view of the first example embodiment of the hand-held advancement apparatus consistent with present principles;

FIG. 6C shows a cutaway view of the first example hand-held advancement apparatus consistent with present principles;

FIGS. 6D and 6E show side cross-sectional views of the first example hand-held advancement apparatus consistent with present principles;

FIG. 6F shows a partial cutaway view of the first example hand-held advancement apparatus consistent with present principles;

FIG. 6G shows a detailed cutaway view of certain portions of the first example hand-held advancement apparatus consistent with present principles;

FIG. 6H shows a side cross-sectional view of the first example hand-held advancement apparatus and a martini medical device in front of the hand-held advancement apparatus as advanceable along a guide wire by the apparatus, along with a table indicating various example wire pullout forces to inform selection of springs embodied in the martini medical device and apparatus consistent with present principles;

FIG. 6I shows another exploded partial view of the first example hand-held advancement apparatus consistent with present principles;

FIGS. 6J and 6K show side cross-sectional partial views of the body and nose portion of the first example hand-held advancement apparatus consistent with present principles;

FIG. 6L shows a top cross-sectional partial view of the aforementioned body and nose portion consistent with present principles;

FIGS. 6M-6P show various side cross-sectional views of the first example hand-held advancement apparatus with the nose portion in various locked and unlocked configurations with respect to the body consistent with present principles;

FIG. 7A shows an isometric view of a first example guide wire alignment device that may be used consistent with present principles;

FIG. 7B shows a side cross-sectional view of the first example cannulated screw alignment device consistent with present principles;

FIG. 7C shows a transverse cross-sectional view of the first example cannulated screw alignment device consistent with present principles;

FIGS. 7D and 7E show isometric views of the first example cannulated screw alignment device as used in combination with a martini medical device during provisional fracture alignment and reduction consistent with present principles;

FIGS. 7F and 7G show additional side cross-sectional views of the first example cannulated screw alignment device to facilitate certain surgical guide wire positionings consistent with present principles;

FIG. 8A shows an isometric view of a second example cannulated screw alignment device that may be used consistent with present principles;

FIG. 8B shows an isometric view of the second example cannulated screw alignment device as used in combination with a martini medical device during provisional fracture alignment and reduction consistent with present principles;

FIG. 9A shows an isometric view of the first example cannulated screw alignment device along with a wire guide and drill guide that may be nested within a chamber of the first example cannulated screw alignment device consistent with present principles;

FIG. 9B shows a side cross-sectional view of the first example cannulated screw alignment device with the wire guide and drill guide nested therein consistent with present principles;

FIG. 10A shows a side elevational view of an example extension rod that may be used during provisional fracture alignment and reduction consistent with present principles;

FIG. 10B shows an isometric view of the example extension rod with two portions of its body being unscrewed from each other consistent with present principles;

FIGS. 10C-10F illustrate different ways the example extension rode may be used with a martini medical device during provisional facture alignment and reduction consistent with present principles;

FIG. 11A shows a front isometric view of a second example embodiment of a martini medical device consistent with present principles;

FIG. 11B shows a rear isometric view of the second example martini medical device consistent with present principles;

FIG. 11C shows a side cross-sectional view of the second example the martini medical device consistent with present principles;

FIG. 12A is a side orthogonal view of a third example embodiment of a martini medical device consistent with present principles;

FIG. 12B is a top orthogonal view of the third example martini medical device consistent with present principles;

FIG. 12C is a cross-sectional side view of the third example martini medical device consistent with present principles;

FIG. 13 shows a side isometric view of a fourth example embodiment of a martini medical device consistent with present principles;

FIGS. 14A and 14B show partial cross-sectional views of alternate nose portions for a martini medical device consistent with present principles;

FIG. 15A shows a side isometric view of fifth example embodiment of a martini medical device consistent with present principles;

FIGS. 15B and 15C show elevational side views of the fifth example martini medical device consistent with present principles;

FIGS. 16A and 16B show side cross-sectional views of a sixth example embodiment of a martini medical device consistent with present principles;

FIGS. 17A and 17B show side cross-sectional views of a seventh example embodiment of a martini medical device consistent with present principles;

FIGS. 18A and 18B show side cross-sectional views of an eighth example embodiment of a martini medical device consistent with present principles;

FIGS. 19A and 19B show side cross-sectional views of a ninth example embodiment of a martini medical device consistent with present principles;

FIG. 20 shows an elevational view of an innovative guide wire that may be used consistent with present principles;

FIG. 21 shows other example guide wires that may be used consistent with present principles;

FIG. 22 shows an example method in flow chart format for surgically using the medical devices and apparatuses described below consistent with present principles;

FIG. 23 shows an example method in flow chart format for manufacturing the medical devices and apparatuses described below consistent with present principles;

FIG. 24 shows an example method in flow chart for providing the medical devices and apparatuses described below consistent with present principles;

FIGS. 25-28 show various example use cases for the martini medical devices disclosed herein consistent with present principles;

FIG. 29A shows a top plan view of a first example tissue retractor that may be used consistent with present principles, while FIG. 29B shows a perspective view of the first example tissue retractor being used with a martini device during provisional fracture reduction consistent with present principles;

FIGS. 30 and 31 show top plan views of second and third example tissue retractors that may also be used consistent with present principles; and

FIGS. 32 and 33 show isometric views of fourth and fifth example tissue retractors that may be used consistent with present principles.

### DETAILED DESCRIPTION

Disclosed below are medical devices that allow compression of tissue and/or implants along a k-wire, Steinmann pin, and/or other alignment mechanism during provisional reduction of fractures in fracture fixation surgery for a human patient. Refined pressure of a desired amount may be applied using these devices, with the pressure being visually and tactilely demonstrated through one example “martini” device's sprung nose and force gauge. A surgeon or other physician may thus verify an intended bone alignment while reducing impact on the bone during provisional reduction, also minimizing significant bone loss that might otherwise occur prior to permanent reduction fixation. Accordingly, the example martini devices discussed below may establish a movable bead or tack that can lock at any point along a k-wire, Steinmann pin, or other structural alignment element due to an innovative collet/camming design, which may take an input force and create a force multiplier that cinches/bites into the wire harder and harder as force to withdraw the martini device from the wire continues to be applied (e.g., absent use of a release mechanism that may be used for withdrawal of the device from the wire as discussed further below).

Also discussed below are hand-held martini device advancement apparatuses that may be used to smoothly and efficiently advance a martini device down a surgical guide wire without pulling the anchor of the wire away from the patient's bone.

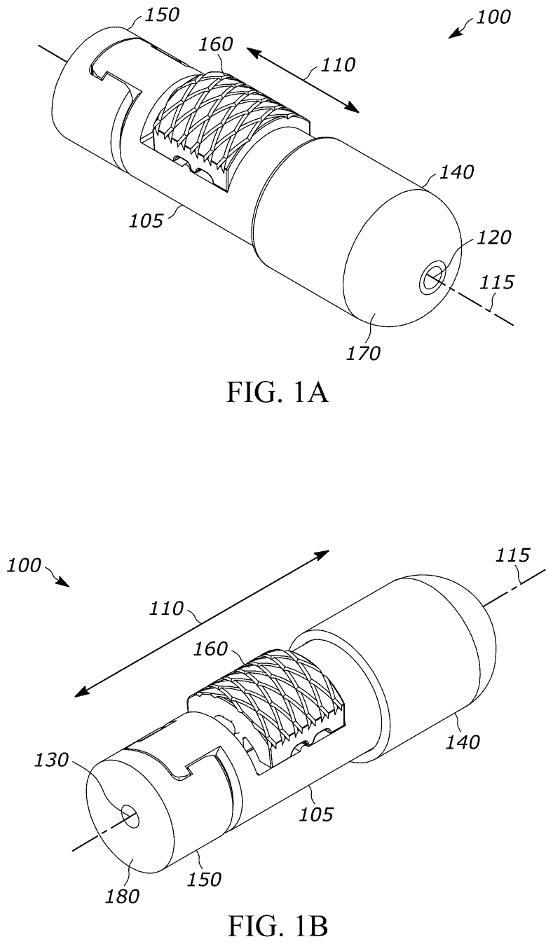

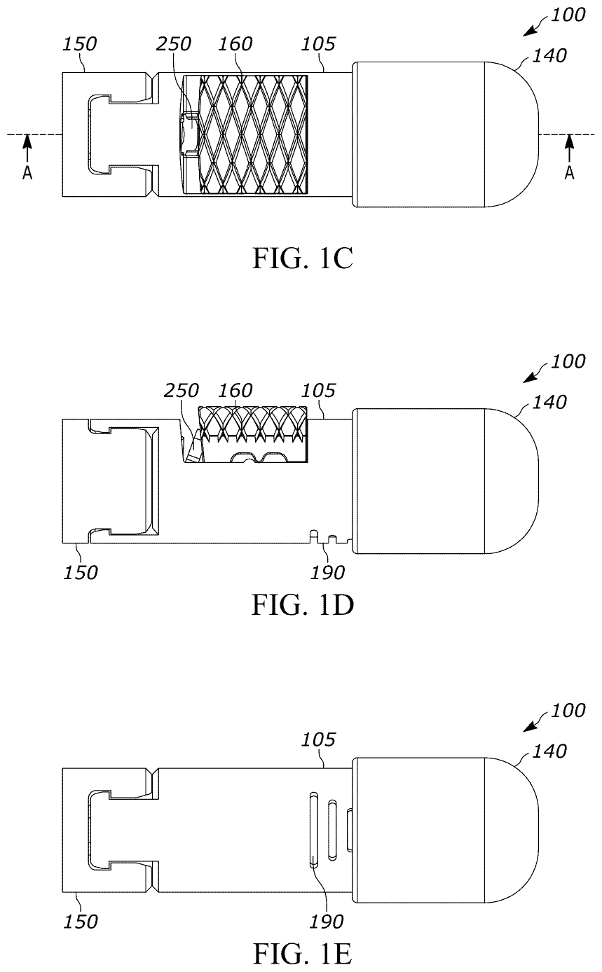

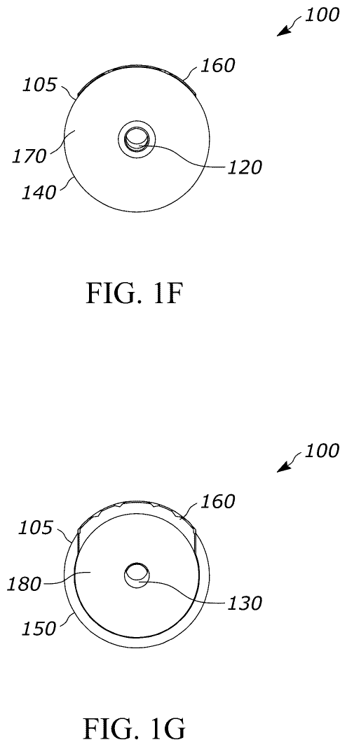

Beginning now in reference to FIGS. 1A and 1B, a first example embodiment of a martini medical device 100 is shown respectively in front and rear isometric view. The martini device 100 may be used consistent with present principles to provide an input force at the nose of the device 100 and create a force multiplier at an aperture 260 of a cam plate 250 (equivalently, cam washer) as described further below to cinch or bite down on a surgical guide wire 115, holding/locking the device 100 in place on the wire 115.

Accordingly, as shown in these figures, the medical device 100 may include a housing 105 with an elongated, rigid body. The housing 105 may be made of metal such as medical-grade steel or aluminum, for example. Additionally or alternatively, the housing 105 may be made of hard plastic, hardened polymer, and/or other suitable material. As also shown in these figures, the housing may define a longitudinal axis 110.

The housing 105 may be at least partially cylindrical as shown to avoid unintentionally catching on other body tissue in the area of the surgical site. The housing 105 may include a first end portion (“nose portion”) 140 of a first diameter as well as a second end portion 150 of a second diameter less than the first diameter. However, note that in other embodiments, the first and second diameters may be the same, or the second diameter may be more than the first diameter.

FIGS. 1A and 1B also show that in non-limiting examples, a first distal external surface 170 of the first end portion 140 may be rounded to establish a convex first end of the housing 105, with the first distal external surface 170 and/or portion 140 more generally including a first aperture 120. These two figures also show that a second distal external surface 180 of the second end portion 150 may be flat in a transverse plane perpendicular to the longitudinal axis 110, with the second distal external surface 180 and/or portion 150 more generally including a second aperture 130. However, in other non-limiting examples both distal external surfaces 170, 180 may be convex, or may be flat. Further note that the first and second distal external surfaces 170, 180 may face outward away from each other as shown.

The external surface(s) may be convex and rounded in non-limiting examples to prevent the device from inadvertently catching or grabbing other things in the surgical environment, which could in turn harm the patient. Also to prevent this from occurring, the exterior surfaces of the housing 105, including the surfaces 170, 180, may be smooth and/or have a polished finish.

FIGS. 1A and 1B also show that the apertures 120, 130 may be circular in height and width and cylindrical in depth. The apertures 120, 130 may have the same or different height/width diameters as each other. The first aperture 120 may therefore have a first height and first width establishing a first plane perpendicular to the longitudinal axis 110, and the second aperture 130 may have a second height and a second width establishing a second plane perpendicular to the longitudinal axis 110. In non-limiting examples, the first and second planes may be parallel to each other. The apertures 120, 130 may help constrain the surgical guide wire 115 as it extends through the device 100, as illustrated by FIGS. 1A and 1B.

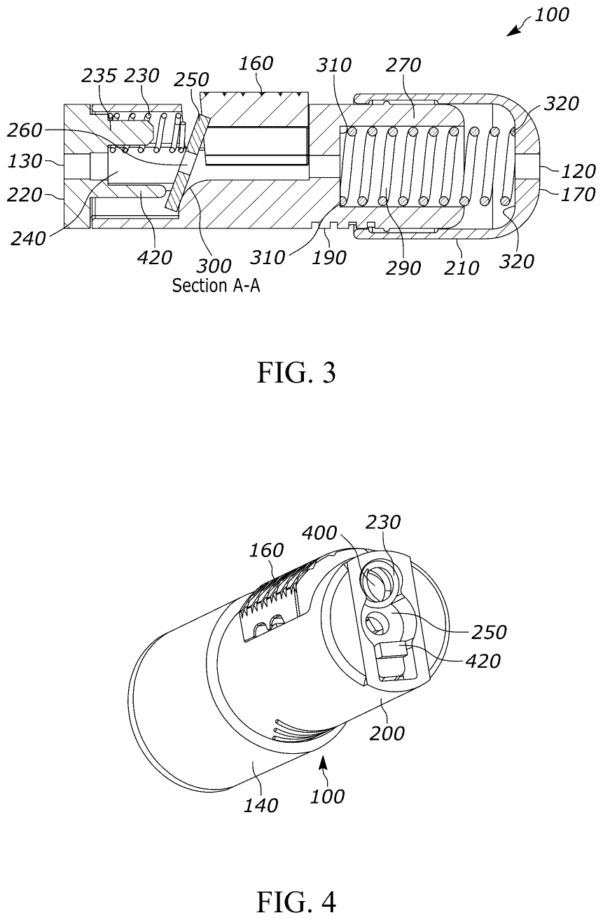

During provisional fracture reduction, the device 100 may therefore receive the surgical guide wire 115 through the first aperture 120, with the wire 115 then being advanced through a hollow channel 240 and aperture 260 in the plate 250 (shown in the cutaway view of FIG. 3) to subsequently exit through the second aperture 130 such that the wire 115 then extends longitudinally through the entire device 100. Owing to the oblique angle of the aperture 260 as described in further detail below, the device 100 may then continue to be advanced down the wire 115 but may not be withdrawn from the wire 115 the opposite way save for manipulation of a release mechanism on the device 100.

Accordingly, FIGS. 1A-3 show one example release mechanism 160 that may be coupled to the housing 105, with the release mechanism 160 being a slider/button in this non-limiting example. The release mechanism 160 may be manipulable to move the metal or polymer plate 250 (or more generally, a structural element 250) inside the housing 105 about a fulcrum 300, with the fulcrum 300 shown best in FIG. 3. This action counteracts a spring bias/biasing moment exerted by a spring 230 inside the housing 105, permitting withdrawal of the surgical guide wire 115 from the third aperture 260 through the first aperture 120 so the device 100 may be removed from the wire 115 from the same direction from which it was advanced.

To further illustrate various aspects of the device 100, note that FIG. 1C shows the device 100 in top orthogonal view, while FIG. 1D shows the device 100 in side orthogonal view. FIG. 1E shows the device 100 in bottom orthogonal view. FIG. 1F shows the device 100 in front orthogonal view. FIG. 1G shows the device in rear orthogonal view.

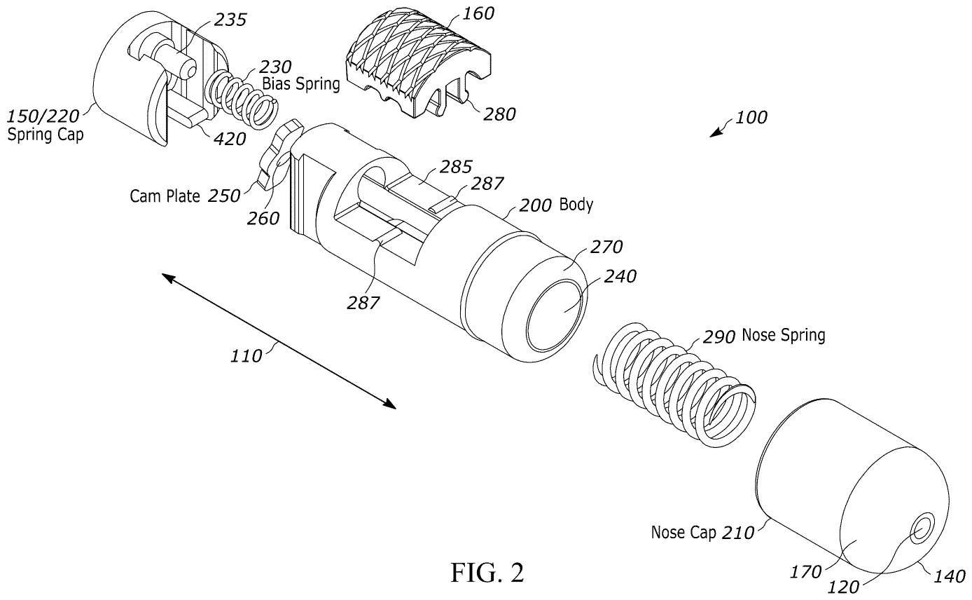

Additionally, FIG. 2 shows the device 100 in exploded view. As may be appreciated from this figure, the housing 105 includes a device body 200, front hollow nose cap 210, and rear spring cap 220. In non-limiting examples, the nose cap 210 may define some or all of the front end portion 140, while the rear spring cap 220 may define some or all of the rear end portion 150.

As also shown in FIG. 2 and further illustrated in the cross-sectional longitudinal side view of FIG. 3 (showing the device 100 as assembled), the device 100 may include the aforementioned cam plate 250 disposed within the housing 105. The cam plate 250 may include the third aperture 260. The third aperture 260 may have a third height and a third width establishing a third plane, where the third plane is oblique with respect to the longitudinal axis 110 while the plate 250 is under spring bias from the spring 230 in the housing 105. The oblique angle of the third plane (e.g., when the plate 250 is at rest under spring bias from the spring 230) may be between eighty five and twenty degrees relative to the longitudinal axis 110 in various non-limiting embodiments, and preferably eighty to fifty degrees and even sixty degrees in particular in specific non-limiting examples. Thus, as best shown in FIG. 3, the first aperture 120, second aperture 130, and third aperture 260 may be at least partially aligned for the surgical guide wire 115 to concurrently extend through the first, second, and third apertures while the plate 250 is still under spring bias from the spring 230 and obliquely oriented. The hollow channel 240 as shown in FIG. 3 may thus extend longitudinally through the transverse center of the housing 105 to fluidly connect the apertures 120, 130, and 200 for the wire 115 to concurrently extend through all three apertures and the channel 240 itself (despite the third plane of the third aperture 200 being oblique with respect to the longitudinal axis and hence not parallel to the first and second planes of the first and second apertures).

Describing the spring 230 in more detail, the spring 230 may be a compression spring (e.g., helical or conical) to oppose compression along the spring's longitudinal axis. However, other types of springs may also be used (e.g., leaf springs), and for that matter other types of structural elements structurally configured for material bias may also be used in addition to or in lieu of a spring. For example, a semi-rigid polymer may be configured in a particular bowed shape to also exhibit a desired bias. But regardless of whether a spring or other type of biased structural element is used, note that the bias may be toward the first end portion 140 such that the spring/structural element 230 resists force/compression from the plate 250 toward the rear end portion 150. To this end, note that the distal segment of the spring 230 may be mounted onto a post 235. The post 235 may be made integral with the cap 200 and extend longitudinally within the housing 105 (e.g., parallel to the longitudinal axis 110). The proximal segment of the spring 230 may then be configured within the housing 105 to abut and impose the spring bias on the plate 250 at a first (upper) area of the plate 250 to help maintain the oblique angle of the third plane with respect to the longitudinal axis 110 and to therefore also impede withdrawal of the surgical guide wire 115 from the third aperture 260 toward the first aperture 120 while the surgical guide wire 115 extends through the third aperture 260.

FIG. 4 is a rear isometric view that further illustrates, with it being noted that the rear spring cap 220 has been omitted to show the first area 400 of the plate 250 mentioned above (the area against which the spring 230 imposes the spring bias to help maintain the aforementioned oblique angle of the third plane). It may also be appreciated from FIG. 4 that the example plate 250 has a generally circular shape in the plate center that itself defines the third aperture 260. The plate 250 also has tabs extending up and down as shown. The upper tab establishes some or all of the first area 400 as generally facing toward the rear of the device. Additionally, the lower tab establishes some or all of a second area of the plate 250 as generally facing toward the front of the device 100. The second area is thus configured within the housing 105 to rest against the fulcrum 300 mounted or made integral with the housing 105. Accordingly, referring back to FIG. 3 for a moment, note that the fulcrum 300 may be established by an inner portion of the body 200 that is rounded from vertical to horizonal, device back to device front. However, further note that other fulcrum configurations are also encompassed by present principles.

Also note per FIG. 4 that the plate 250 is constrained not just by the fulcrum 300 but also by inner sidewalls of the housing 105 establishing an opening for the plate 250. This opening may therefore be shaped like the plate itself but may be slightly larger than the plate 250 to closely receive the plate 250 and constrain it from jostling sideways and up/down in an X-Y plane of the device 100 (the X-Y plane being perpendicular to the longitudinal axis and cutting transversely through the device 100). However, owing to their configuration, these cam plate constraint features still allow controlled radial/rotational movement of the plate 250 about the fulcrum 300 in the Z dimension. To further constrain the plate 250 while allowing this controlled movement, a longitudinally-extending structural element 420 on the cap 220 may abut the rear-facing portion of the lower tab of the plate 250, where the elements 420 and 300 form a pocket or hinge helping to maintain alignment of the apertures 120, 260, 130 even when the plate 250 is obliquely oriented.

It may be further appreciated from these figures that the plate 250 is configured within the housing 105 to rest against the structural element 420 and counteract the bias from the spring 230 during advancement of the wire 115 through the third aperture 260 from the direction of the first aperture 120. This plate resting against the element 420 during advancement is effected due to the friction force that is created between the wire 115 and plate portions around the aperture 260 as the device 100 is advanced down the wire 115. Accordingly, the friction force rotates the third plane of the third aperture 260 closer to parallel with the first and second planes to the first and second apertures 120, 130.

Based on the foregoing, it is to be even further understood that when the release mechanism 160 is activated, the release mechanism 160 unloads the plate 250/wire interface, with the plate 250 rotating within the pocket formed by fulcrum 420 and 300 to become more parallel. Thus, fulcrum 420 and 300 may both contain the plate 250 in the device 100 and form a point of rotation for plate rotation (e.g., when the release mechanism is active, moving the plate 250 closer to parallel). Thus, if the device 100 is holding load, that load is acting through fulcrum 300. Then to release the load, first the spring 230 is compressed until the plate 250/wire interface releases and then the plate may rotate and slide against the fulcrum 300. Accordingly, the structures of the elements 300 and 420 may together form a pocket or hinge point within which the plate can rotate.

Referring back to FIGS. 2 and 3 and further describing this pocket or hinge point created by the fulcrum 300 and element 420 of the device 100, the hinge point could instead be created by a pin and hinge assembly where a hole is formed cross-wise in the end near the second area of the plate 250 with an axis of this hole perpendicular to the longitudinal axis 110 of the device 100 and another hole formed in the body 200 such that it may be substantially aligned and concentric with the hole formed in the end of the plate 250. A pin made of metal or hard plastic or other substantially rigid and strong material may be positioned and assembled through these aligned holes in the plate 250 and body 200 to couple these components together and form a pinned-hinge connection.

Still referring to FIGS. 2 and 3 and describing other aspects of the device 100, the first end portion 140 of the device 100 may also include telescoping members that slide with respect to each other according to the longitudinal axis 110. In the present example, the nose cap 210 establishes one of the telescoping members and has a larger diameter than a second telescoping member 270 on the body 200 (though in other example embodiments the nose cap 210 may have a smaller diameter than the member 270 and telescope inside the body 200/member 270). The first telescoping member 210 is therefore distal to the plate 250 while the second telescoping member 270 is proximal to the plate 250.

As also shown in FIGS. 2 and 3, the first telescoping member 210 may be configured to slide toward the plate 250 to counteract bias from and compress a nose compression spring 290 on (e.g., in) the housing 105 that exerts force on the first telescoping member 210 to push the first telescoping member 210 distally away from the second telescoping member 270. The cross-sectional view of FIG. 3 thus illustrates that when the device 100 is assembled, the spring 290 extends longitudinally within the housing 105 so that it is coaxial with or at least parallel to the longitudinal axis 110, abutting one or more proximal walls 310 in the body 200 at the proximal end and abutting the inside front walls 320 of the hollow nose cap 210 at the distal end. Further note that the inside of the spring 290 may help establish some of the channel 240.

However, note that in other embodiments, the spring 290 may be located outside the housing body 200/member 270 such that the inner diameter of the spring 290 is greater than the outer diameter of the distal end of the housing (200/270). Additionally, in some examples the spring 290 may be integral with the housing 200/270.

Also note in terms of the distal nose cap 210 that it may be attached to the member 270 using a ring/rib on the external surface of the member 270 (circumscribing a transverse segment of the member 270) such that the nose cap 210 may be snapped over the ring/rib to couple to the member 270. The nose cap 210 may also have a relief region (e.g., of a greater diameter than the member 270) that allows telescoping movement but keeps the nose 210 attached to the body 200/270 such that it cannot slide distally off past the ring/rib.

The telescoping members 210, 270 and nose spring 290 are thus configured to reduce and absorb backlash in cam plate engagement with the wire 115—and increase compression against the bone or plate as the spring 290 pushes the body 200 backwards away from fracture site (and the cam plate 250 thus bites down on the wire 115 even harder due to force from the spring 290)—since slight wire travel within the device 100 can occur before the wire 115 gets cinched/bound in the third aperture 260 after advancement to a desired wire location. And to reiterate, once at the desired wire location, the device 100 may help maintain a compression force along the wire 115 between bone fragments and/or plates for ascertaining proper bone and/or plate alignment during fracture reduction or other bone repair (e.g., before much larger holes are drilled into the bone to insert screws or other fasteners for permanent fixation).

Thus, in one example, a surgeon may advance the device 100 up against the plate or bone, nose portion 140/cap 210 first. With the device 100 obstructed by the plate/bone at the front of the nose cap 210/device 100, additional advancement of the device 100 from the surgeon toward and against the plate/bone may thus result in compression of the spring 290 until the maximum telescoping range of motion of the front end portion 140/nose cap 210 with respect to the body 200 is reached, resulting in the maximum spring 290 compression force. Once the surgeon releases his/her manual force from device 100 as applied through the aforementioned additional advancement, the compression spring 290 pushes against the nose cap 210 and into the body 200, moving the body 200 backwards away from both the nose cap 210 and the bone/plate itself while the cap 210 remains up against the bone/plate. This force translates through the fulcrum of body 200 and into cam plate 250, resulting in an increasing moment about the contact points between the wire 115 and the through hole 260 to lock the body 200 on the wire 115 (e.g., such that the body 200 cannot move backward any additional distance relative to wire 115 due to the plate 250 becoming more obliquely oriented as the body 200 moves backward away from the nose cap 210 due to the spring force, locking the body at that point on the wire 115). The spring 290 force is then maintained between the wire/cam plate interface and the nose cap 210 with the cap 210 still obstructed by the plate/bone. This, in turn, results in the device 100 using the anchored wire 115 to apply a compressive force through spring 290 to the bone/implant and even through the aligned (fractured) bones.

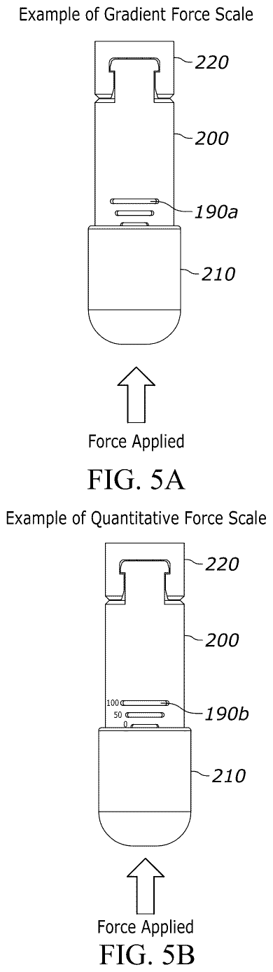

A force gauge 190 as also shown in various figures may further aid the physician in this task. In one example, the gauge 190 may be established by notches in an external surface of the body 200, where those notches provide an indication of the amount of force between cap 210 and body 200 due to the bias from the spring 290. Example force gauges 190a and 190b will be described in greater detail later in reference to FIGS. 5A and 5B.

But still in reference to FIGS. 2 and 3 and describing the one or more release mechanisms 160 in more detail, again note per the example shown that the mechanism(s) 160 include a slider button as shown. To slide longitudinally along the housing 105, the slider 160 may have a female track 280 mountable on a male track 285 on the body 200 to constrain the slider 160 from being removed up away from the body 200 and transversely across the body 200, while still permitting longitudinal movement along the combined track 280/285. Accordingly, when the device 100 is assembled as shown in FIG. 3, the rear portion of the slider 160 may abut the upper tab of the plate 250 such that a physician may take his/her finger(s) and slide the slider 160 back to rotate the plate 250 about the fulcrum 300. This action counteracts the spring bias from the spring 230 to bring the third plane of the third aperture 260 closer to parallel with the first and second planes to permit the wire 115 to be removed/withdrawn out of the first aperture 120 unencumbered by some or all of the cinch/binding action caused by the oblique angle of the third aperture 260 itself while the wire 115 extends through the third aperture 260. Also note that owing to the plate 250 having bias from the spring 230 exerted against it as described above, the slider 160 is also biased in a similar manner absent the physician sliding it back. However, further note that in some non-limiting examples, transverse ridges or ribs 287 on the upper portion of the track 285 may also receive reciprocal notches on the lower portions of the slider track 280 to provide some friction force to help maintain the slider 160 at a desired position on the track despite spring bias.

For completeness and before moving on to other figures, note that one or more of the body 200, cap 210, and/or cap 220 may be made integrally with each other, and/or may be engaged via snap fit, adhesive, etc. Either way, once coupled together, these components help to constrain the inner parts of the device 100 to bind and release the wire 115 from the third aperture 260 as desired to help with alignment of bone(s) (such as two bone fragments/segments of a same bone structure like a radius that is to be integral, absent fracture) and/or surgical plates prior to permanent fixation to ensure proper alignment before said permanent fixation.

Also before moving on to description of other figures, it is reiterated that while the first aperture 120, second aperture 130, and third aperture 260 may be circular as shown in the figures described above (with the respective height and width of each aperture both being measures of the respective diameter of the respective aperture itself), in other examples one or more of these apertures may be shaped differently if desired and depending on implementation. For example, one or more of the first, second, and/or third apertures may be oblong instead (e.g., oval-shaped with a long axis transverse to the longitudinal axis 110 sideways across the housing 105).

Now in reference to FIGS. 5A and 5B, two example implementations of the aforementioned force gauge 190 are shown, both of which may indicate an amount of force the second spring 290 exerts on the first telescoping member (cap 210). FIG. 5A shows an example gradient force scale/gauge 190a with line markings of increasing width for progressively increasing force, while FIG. 5B shows an example quantitative force scale/gauge 190b with increasing numbers for progressively increasing force (e.g., from zero to 100 Newtons in the present example).

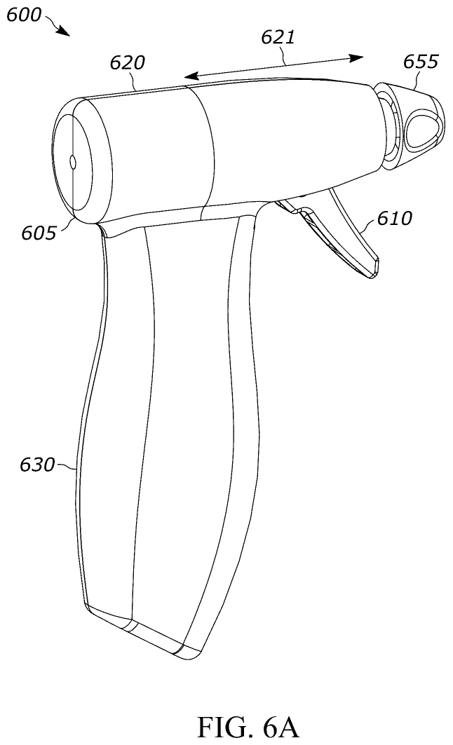

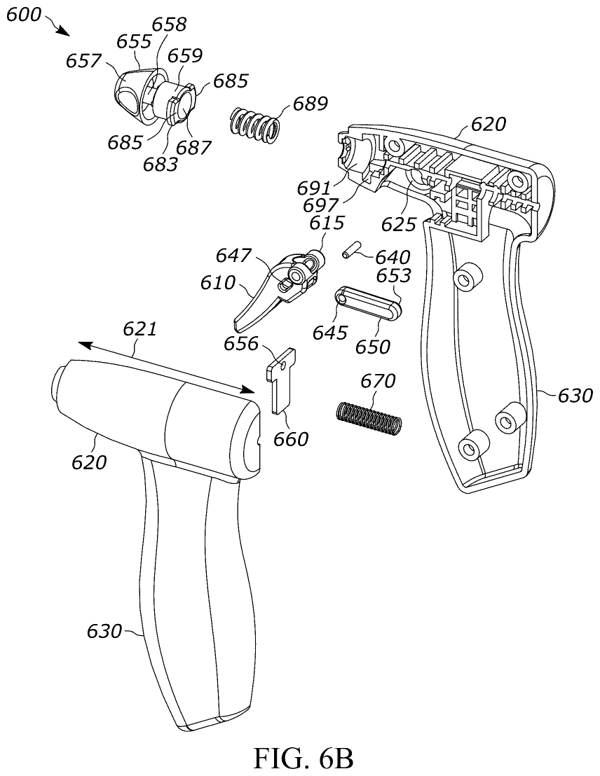

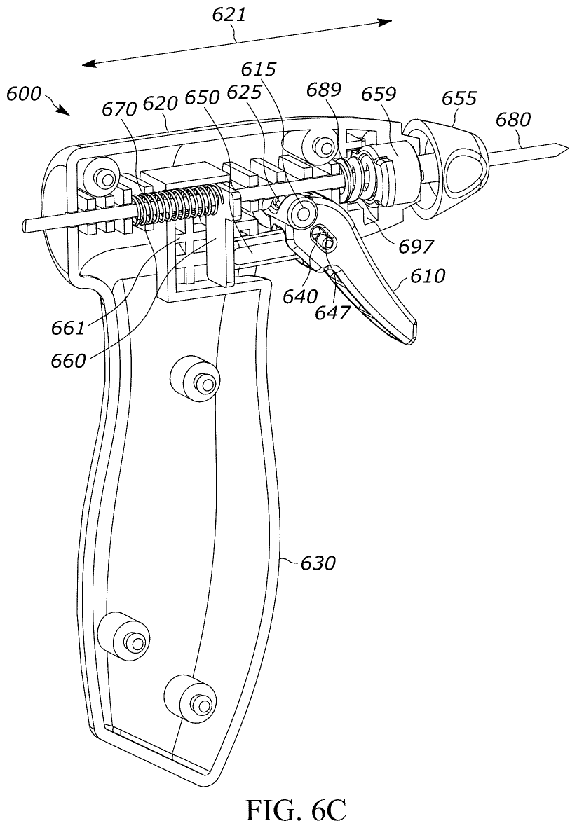

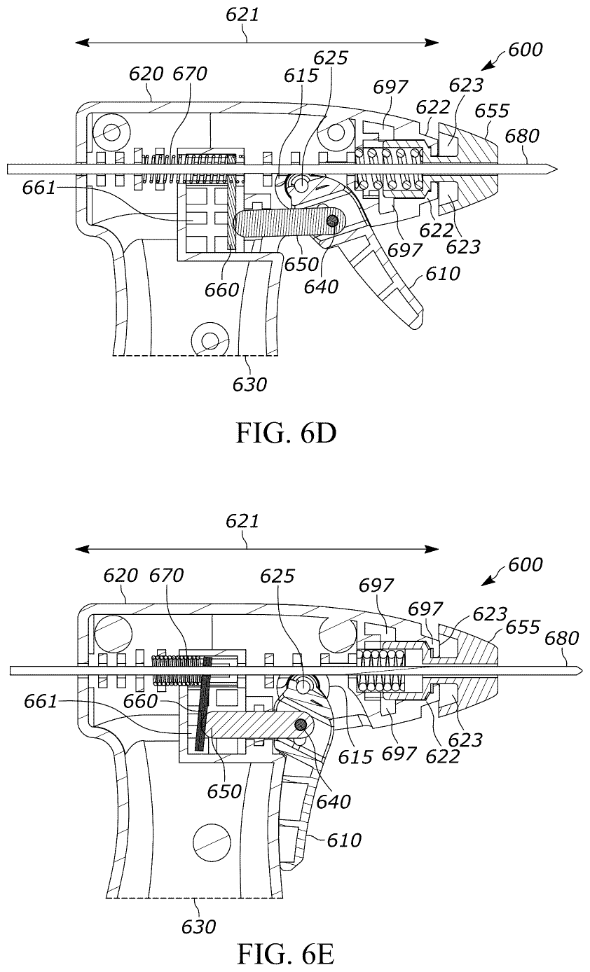

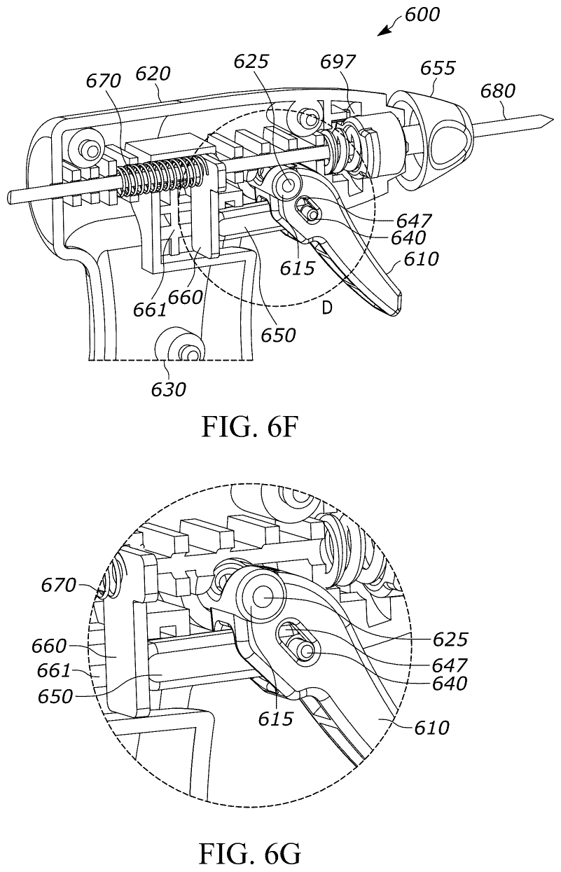

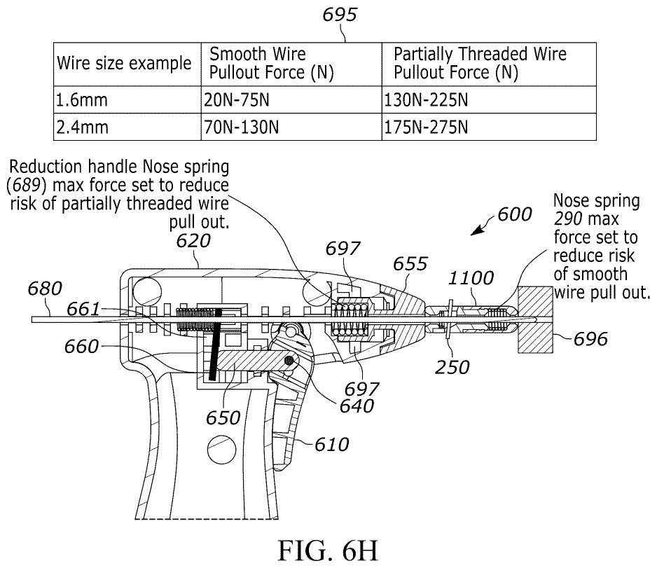

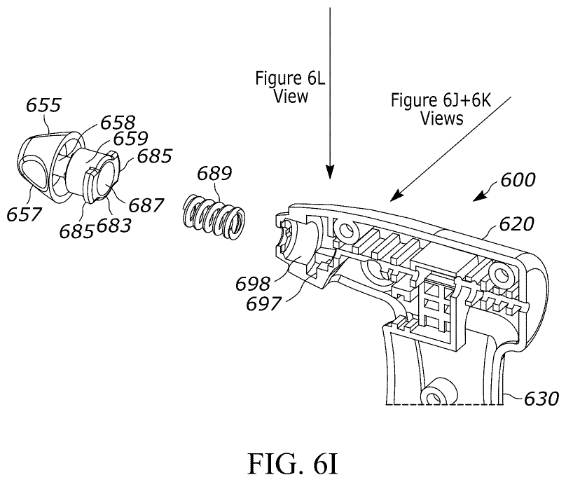

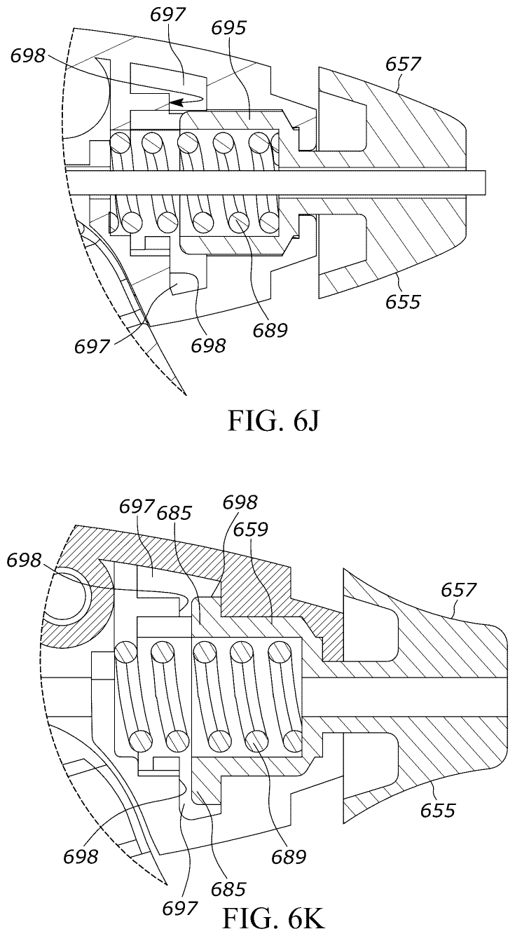

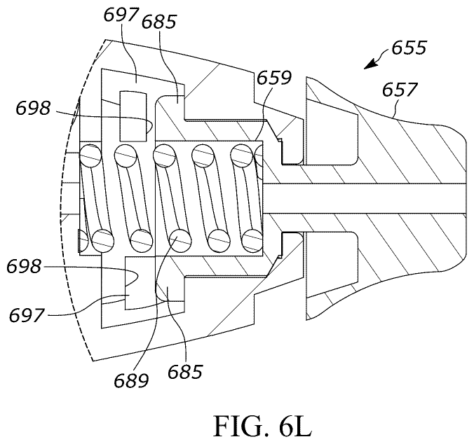

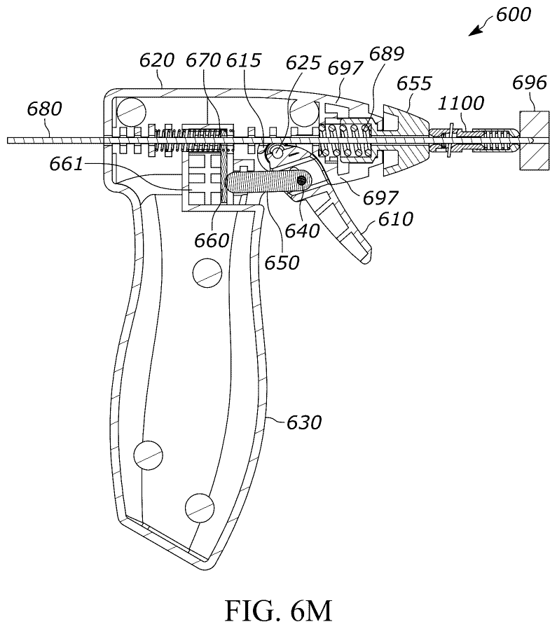

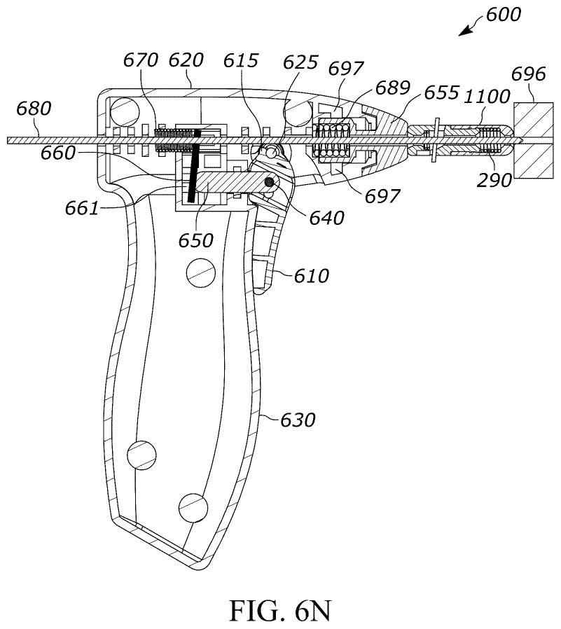

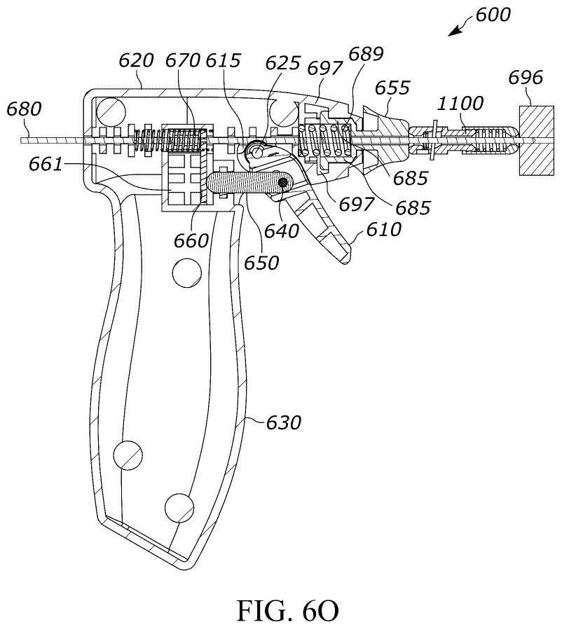

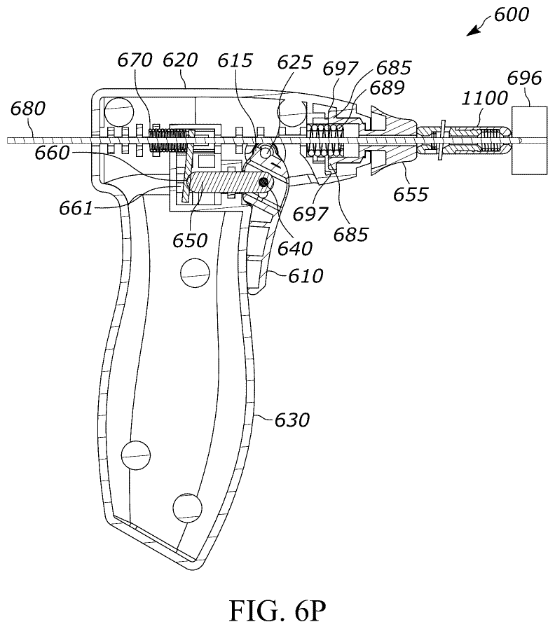

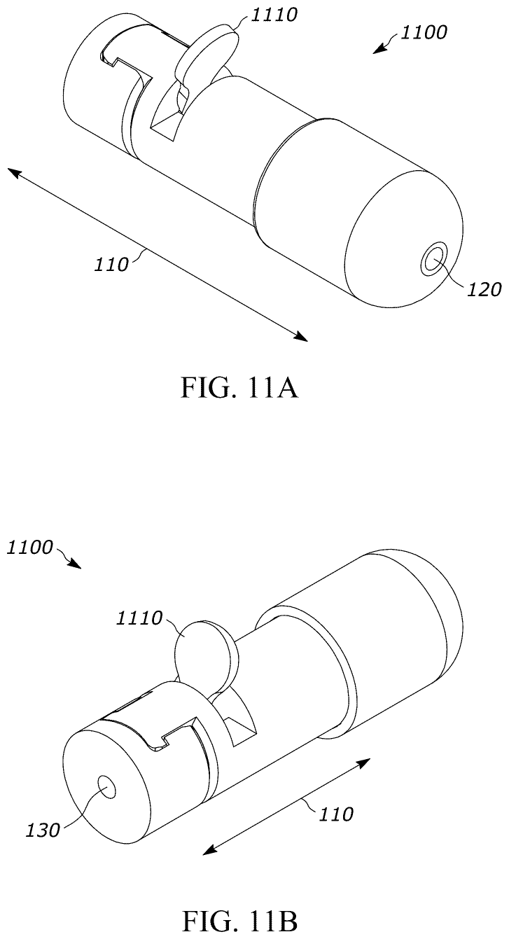

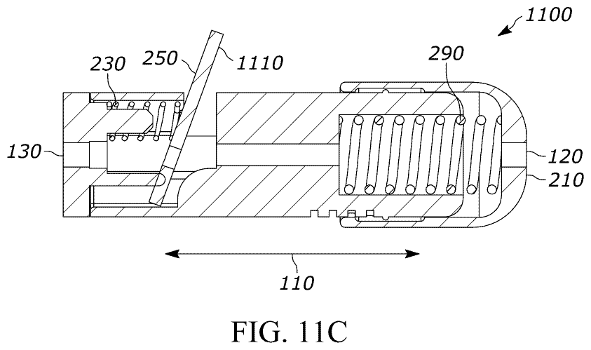

Continuing the detailed description in reference to FIGS. 6A-6H, a first example hand-held martini device advancement apparatus 600 (sometimes referred to as a reduction handle) is shown. The apparatus 600 may be used to advance a martini device 1100 down a k-wire 680 toward a fracture site during provisional alignment and fracture reduction consistent with present principles, with it being further noted that the martini device 1100 may be the same as the device 100 save for having a lever as its release mechanism rather than the slider 160. The martini device 1100 will be described in greater detail below in reference to FIGS. 11A-11C.

In particular reference to FIGS. 6A-6C, note that FIG. 6A shows an isometric view of the apparatus 600, FIG. 6B shows an exploded view of the apparatus 600, and FIG. 6C shows a cutaway view of the apparatus 600. The apparatus 600 may establish a compression clamp assembly that may be used to help advance the device 1100 along the surgical guide wire 680 as threaded through the device 1100 as well (rather than advancing purely by hand as also encompassed by present principles). The apparatus 600 may therefore be helpful as it can be used to advance the device 1100 along the guide wire 680 in a more controlled manner, potentially while also using less hand force than advancement by hand alone due to the leverage provided by a mechanism (including cam plate 660 or, equivalently, cam washer) in the apparatus 600 that cinches or otherwise grips the guide wire 680 to move the wire 680 from front to back through the apparatus 600 to advance both the apparatus 600 and martini device 1100 (in front of the apparatus 600) down the guide wire 680. The device 1100 may therefore be advanced on the guide wire 680 using a pull force implemented by the apparatus 600 to push the martini device 1100 forward, whether the apparatus 600 is physically attached to the device 1100 or simply pushes the device 1100 from behind.

Describing the apparatus 600 in more detail, as shown in FIGS. 6A-6C the apparatus 600 may generally be in the shape of a clamp gun. The apparatus 600 may include a housing 605. The apparatus 600 may also include an elongated, rigid front arm/trigger 610 that rotates with respect to an elongated, rigid body 620 of the apparatus 600. The apparatus 600 may also include an elongated, rigid rear arm/handle 630 that intersects and is coupled to the body 620. The handle 630 may be immobile with respect to the body 620. The housing 605 may thus define both the body 620 and handle 630 and, in one particular example, the handle 630 may be made integral with the body 620. The trigger 610 and other mechanical components of the cam plate movement mechanism may be coupled to the body 620 (and/or handle 630) as described in greater detail in a moment. Note that the structural elements 610, 620, and 630 may be made of metal such as medical-grade steel or aluminum, and/or may be made of hard plastic, hardened polymer, and/or other suitable material.

As for the coupling of the trigger 610 to the body 620, this may be implemented in part using a hollow, cylindrical, rigid and elongated upper segment 615 on the trigger 610 that has an opening at the top of the segment 615 to receive a cylindrical, rigid, and elongated pin or protrusion 625 inside the body 620 (e.g., formed in the housing 605) to establish an axis of rotation of the trigger 610 with respect to the body 620. The pin or protrusion 625 may therefore be made integral with or otherwise coupled to the body 620, while the cylindrical upper segment 615 may be made integral with the trigger 610 (or otherwise coupled thereto). The segment 615 may have a longitudinal axis that is orthogonal to a longitudinal axis of the trigger 610 itself, with the longitudinal axis of the segment 615 running transverse through the body 620 when the segment 615 is engaged therewith. Further note that while engaged with the body 620, the pin or protrusion 625 may extend through the hollow interior of the segment 615 for rotation of the segment 615 with respect to the pin 625 according to the aforementioned axis of rotation. The segment 615 and pin/protrusion 625 may each be made of a surgical-grade metal or even a plastic or other polymer, as may other components of the apparatus 600.

FIGS. 6B and 6C also show that a metal or polymer pin 640 (or other structural element) may extend through an opening 645 in a push rod/linkage bar 650 that is configured for coupling to the apparatus 600, with the pin 640 also concurrently extending through openings 647 on the sides of an upper rear portion of the trigger 610 to attach the bar 650 to the trigger 610. With the bar 650 thus coupled within the apparatus 600, a second end portion 653 of the bar 650 (the portion 653 being opposite the other end portion of the bar 650 that bears the opening 645) may abut and even attached to the front face of the cam plate 660 at a vertical distance on the plate 660 that is between 0.5 mm and 12.0 mm below the bottom of the wire 680/bottom of the opening 656 to thus engage the bar 650 with the cam plate 660 within that area of the plate's front face. If desired, the bar 650 may also abut/attach to the plate 660 within that vertical range at a horizontal/transverse middle of the plate 660. This provides desired cam plate action as discussed in greater detail below (including radial and then linear movement) without the plate 660 cinching too hard on the wire 680 so as to bind and bend the wire whilst frustrating the wire's movement through the body 620, as might otherwise occur if the bar 650 pushed on the front face of the cam plate 660 beyond the 12 mm point described above. And further note that if the bar 650 were to push on the front face of the cam plate 660 at a distance less than the 0.5 mm point described above, cinch action might not occur at all.

Additionally, note that as shown in FIG. 6B, the cam plate 660 itself may be in the shape of an upper case “T” in non-limiting examples, with the horizontal bar of the “T” being located in its own track within the body 620 to aid in smooth movement of the cam plate 660 as described in greater detail below. However, other shapes may also be used for the plate 660. But regardless of shape, further note here that the plate 660 may have an opening 665 that may be circular or oblong for extension of the guide wire 680 therethrough so that, as the plate 660 moves to a more oblique orientation with respect to the longitudinal axis 621 due to pullback of the trigger 610 toward the handle 630 (rather than being positioned in an orthogonal/vertical plate orientation with the trigger 610 extended under spring bias), the guide wire 680 gets cinched within the opening 665 similar to how the plate 250 of the device 100 also cinches down on a guide wire as described above. This allows the wire 680 to subsequently be advanced through the body 620 as the trigger 610 continues to be pulled in the same instance.

Thus, as may also be appreciated from FIG. 6C, with the cam plate 660 positioned and constrained transversely within the body 620, the cam plate 660 may first move radially about an axis of rotation at the top of the plate 660 (e.g., at the horizonal bar of the “T” in the separate track for that component) based on actuation of the arm/trigger 610, cinching the wire 680 in the opening 656. Then, as the trigger 610 continues to be pulled during that same trigger pull with the wire 680 already cinched, the plate 660 may move linearly back through an open chamber 661 in the housing 605 (rectangular prism-shaped in non-limiting embodiments) due to the location at which the bar 650 contacts the plate 660 as described above, thereby moving the wire 680 through the body 620 from front to back. Further note that the chamber 661 may have an opening at the top for the lower portion of the plate 660 to extend into the separate track for the horizontal bar of the “T” of the plate 660, with that separate track running longitudinally through the body 620 above the chamber 661 (this track being wider than the chamber 661 itself to accommodate to horizontal bar of the “T”).

Accordingly, the trigger 610 can be pulled back toward the handle 630 to move the cam plate 660 about the upper axis of rotation via the transfer of the trigger force through the bar 650 and to the plate 660 to rotate the plate 660, cinching down on the guide wire 680. From there the plate 660 may, while in an oblique orientation with respect to the longitudinal axis 621 to cinch the wire 680, move linearly backwards to advance the device 600 forward along the wire 680 (pulling the wire 680 through the device 600). Then when the trigger 610 is released, a compression spring 670 exerts a forward force on the plate 660 (this spring force previously being overcome via the trigger pull) for the plate 660 to first stop cinching the wire 680 within the device 600 and then to return the plate 660 to its forward position within the chamber 661, with the device 600 remaining advanced farther down the wire 680 due to the trigger pull.

Also note here that the wire 680, with the trigger 610 released, can then move freely back and forth through the rest of the wire channel in the body 620. But without manual pullback by the physician, at this point the apparatus 600 may still remain advanced down the guide wire 680 due to the previous trigger pull notwithstanding its ability to freely move. Another trigger pull may then be performed to advance the device 600 even farther down the wire 680 and/or to press the martini medical device 1100 up against a bone or plate with the retractable nose portion 655 as will be described later.

With this understanding and to particularly describe movement of the bar 650 itself during trigger pulls and releases, the bar 650 may move substantially parallel to the longitudinal axis 621 of the elongated body 620 (e.g., absolute parallel to parallel within five degrees) from beginning to end of trigger pull and release. Thus, with the pin 640 concurrently extending through the openings 645 and 647 to engage the bar 650 with the trigger 610, the trigger 610 may be pulled back and then released in radial movement. This moves the bar 650 linearly backward toward the handle 630 on pullback of the trigger 610 (moving the bar 650 away from a nose portion 655 of the apparatus 600), and then moves the bar 650 forward upon trigger release (moving the bar 650 back toward the nose portion 655), according to the longitudinal axis 621 of the body 620. The bar 650 is thus pushed forward, pushing the trigger 610 itself forward, on trigger release based on the force exerted on the rear face of the plate 660 by the spring 670.

Stated a bit differently, during a trigger pull, the second end 653 of the bar 650 is pushed against the aforementioned lower area of the front face of the cam plate 660, causing the cam plate 660 to rotate and, in the process, cinch or otherwise grip a particular portion of the guide wire 680 to then pull the wire through the body 620 from front to back as the trigger pull continues. This movement of the plate 660 also counteracts the spring bias exerted on the upper portion of the plate 660 at the opposite (rear) face, with the spring bias being exerted by the compression spring 670. Further note that the rear end of the spring 670 may be mounted to the housing 605, and that the front end of the spring 670 may be mounted to the rear (upper) face of the plate 660.

It may be further appreciated that, as alluded to above, a trigger pull action advances not only the apparatus 600 down the wire 680 while the wire 680 extends through the opening 656 in the plate 660 and is cinched by the plate 660, but also advances (pushes) the martini device 1100 in front of the nose portion 655 down the wire 680 as well (as pushed by the front of the nose portion 655 in particular). To be clear, the wire 680 has already been extended through the martini device 1100 at this point. With the wire 680 then entering the apparatus 600 through a front opening in the nose potion 655, with the nose portion 655 extending distally away from the body 620, for the wire 680 to then extend through a wire channel in the rest of the body 620 as well as through the plate opening 656 as aligned with the wire channel. From there, loose portions of the wire may be extended out another opening in the back of the body 620. With the wire 680 so arranged, the aforementioned trigger pulls may be performed by the physician to concurrently advance both the apparatus 600 and martini device 1100 down the wire in uniform.

The longitudinal cross-sectional partial views of the apparatus 600 as shown in FIGS. 6D and 6E further illustrate the structural components of the apparatus 600 and their relationship to each other. First, it is noted that the distal portion of the body 620 may include teeth 622 that are closely received into openings 623 in the nose portion 655 so that, when the nose portion is fully retracted, the nose portion 655 may be removably secured to the body 620 to prevent unintended movement of the portion 655 with respect to the body.

Additionally, as may be appreciated from FIG. 6D, while the trigger 610 is in the extended position farther away from the handle 630, the cam plate 660 is disposed forward and vertically (or near-vertically) in the chamber 661 under bias from the compression spring 670, concentrically aligning the opening 656 with the channel in the body 620. This allows the k-wire 680 to move freely/unimpeded back and forth (no cinching) through all of the nose portion 655, the body 620 via the wire channel, the opening 656 in the plate 660, and the rear opening in the body 620. Thus, while in this configuration, the X-Y plane of the plate 660 is oriented orthogonal to, or nearly orthogonal to, the longitudinal axis 621 of the body 620.

Then when a physician mechanically actuates the trigger 610, pulling the trigger 610 back toward the handle 630 as shown in FIG. 6E, the plate 660 overcomes bias from the compression spring 670 for the bar 650 to push on the front face of the cam plate 660 on the aforementioned lower area thereof, rotating the plate 660 radially to an oblique position within the chamber 661 to cinch the wire 680. And due to additional trigger movement backwards via the same singular, possibly continual pull action, the plate 660 also moves linearly backwards within the chamber 661 as also shown in FIG. 6E. This advances the wire 680 through the body 620 with one or more portions of the plate 660 around the opening 656 still cinching down on the wire 680 to pull the wire 680 through the device 600.

Overall, it may therefore be appreciated that during provisional alignment and reduction, as the trigger 610 continues to be pulled toward the handle 630, released under spring bias to extend the trigger 610 back toward the nose portion 655, and then pulled anew, the plate 660 cinches at different points on the wire 680 with each pull to progressively move the wire 680 through the body 620. This advances the apparatus 600 itself forward along the wire 680 with each trigger pull (and hence pushes the martini device 1100 forward as located distally in front of the apparatus 600 along the wire 680) so that successive forward portions of the wire 680 progress first through the nose potion 655 and then out the back of the body 620. It is to be further understood that the cinch/bite action on the k-wire 680 hinders the wire 680 from moving the opposite way back out of the body 620 during trigger pull and with the trigger 610 pulled all the way back. The physician may thus incrementally move the apparatus 600 and hence the device 1100 along the wire 680 and toward the fracture site by repeatedly actuating the trigger 610.

Refer now to the cutaway partial view of FIG. 6F and the detailed trigger/body view of FIG. 6G. It may be appreciated from these figures that the opening 647 as located on the upper portion of the trigger 610 may be established by a slot running lengthwise down the upper portion of the trigger 610 according to the longitudinal axis of the trigger 610. The opening 647 may therefore allow the pin 640 to travel up and then down within the opening 647 as the trigger is pulled and then released, respectively. This facilitates smooth radial movement of the trigger about its axis of rotation relative to the body 620 to be translated into smooth linear movement of the bar 650 to then push on the plate 660 to move the plate as described above.

Now refer back to the exploded view of FIG. 6B. As also shown in this figure, the nose portion 655 may have a frustoconical exterior 657 as shown, with the frusto-cone sloping inward proximal to distal. The nose portion 655 may include a first cylinder 658 attached to an inner front wall of the portion 655. The cylinder 658 may be coupled to (e.g., integral with) another cylinder 659 of a larger diameter extending proximally toward the handle 630. The cylinder 659 may be hollow.

As also shown in FIG. 6B, a flange 683 may be located at a proximal end portion of the cylinder 659 nearer the body 620. The flange 683 may define tabs 685 or other protrusions extending transversely away from the cylinder 659, with it being further noted that the tabs 685 may partially but not fully circumscribe the cylinder 659 and, as such, may be located at opposing sides of the proximal end portion of the cylinder 659. The tabs 685 may thus taper or drop off radially to not circumscribe other portions of the cylinder 659.

The tabs 685 will be discussed in greater detail later. But first, as mentioned above, the cylinder 659 may be hollow. The cylinder 659 may also include an opening 687 to the hollow interior to receive a distal portion of a compression spring 689 that extends into the hollow interior of the opening 687 to abut a front inner wall of the cylinder 659. A proximal portion of the compression spring 689 may also be received into an opening 691 in the body 620, with the proximal portion abutting an inner wall inside the body 620 to hold the spring 689 in place between the nose portion 655 and body 620. The spring 689 may thus provide action between the portion 655 and body 620 during physician use, allowing linear movement of the portion 655 with respect to the body 620 along the longitudinal axis 621 to overcome spring bias from the compression spring 689 (spring bias that otherwise applies a force at the spring's distal end to push the nose portion 655 distally away from the body 620). This force may be applied, for example, to advance and securely press the martini device 1100 as located in front of the portion 655 up against a bone or plate during fracture alignment and reduction.

It is to also be understood that, in non-limiting embodiments, the maximum/total travel of the nose portion 655 from its fully-extended position backwards toward the body 620 to thus assume a fully-retracted position may be less than or equal to the maximum/total travel of the plate 660 within the apparatus 600 during trigger pulls. This allows the nose portion 655 to push backwards toward the body 620 against bias from the spring 689 when the martini device 1100, as already contacting the front of the portion 655, is itself pressed against and therefore obstructed by the bone, plate, or other surface during a trigger pull. This helps secure a desired fracture alignment while preventing the body 620 from further advancing along the wire in a way that might pull the wire's anchor out of the patient's bone.

However, in other non-limiting embodiments, the travel of the nose cone 655 may be greater than the travel of the plate 660. But still, a full trigger pull when the nose cone 655 is obstructed by the bone/plate may result in a targeted limitation of pull on the wire. Thus, here the travel of the nose spring 689 may still be calibrated for the device 600 to actuate and compress the spring 689 when advancement is obstructed by the bone/plate, thus reaching a final (fully-retracted) nose position with respect to the body 620 that provides a desired maximum spring force. In this way, a mechanical stop is still provided, providing a known travel that can be related to a spring rate and ultimately determine a max force.

Then when the trigger 610 is released, the body 620 may move backward on the wire 680 while the portion 655 remains in place due to free movement of the wire 680 through the opening 656 responsive to trigger release. Thus, the apparatus 600 is prevented from continuing to try to advance along the wire 680 with subsequent trigger pulls once the nose portion 655 and martini device 1100 reach the bone/plate with no more space to travel, which again might otherwise pull the anchored portion of the wire 680 away from the anchor point on the bone and harm the patient.

With this in mind, it is to be further understood that the linear compression force of the spring 689 may be set just below the amount of force it would take to pull out a threaded k wire that is anchored to the bone site during fracture reduction. This aids in preventing the wire 680 from pulling away from the bone to which it is secured while the plate 660 grips (e.g., bites down) on the wire 680 and nonetheless tries to force the apparatus 600 forward (and hence martini device 1100 forward) as described above. Again, this is advantageous as pulling the wire 680 away from the bone would be counterproductive to fracture reduction as the bone anchor of the wire pulls away from the bone.

For greater understanding, refer to now FIG. 6H. This figure shows both a cross-sectional view of the devices 1100 and 600 as aligned together the wire 680 once they reach a bone/plate 696. This figure also shows a table 695 of example non-limiting wire pullout force amounts that inform selection of compression force values for which the spring 689 of the apparatus 600, and for which the nose spring 290 of the martini device 1100 itself, may be set to avoid the aforementioned pulling of the wire away from the bone/plate 696 when the apparatus 600 advances and presses the device 1100 up against the bone/plate 696 via actuation of the trigger 610.

As shown in FIG. 6H, the trigger 610 has been pulled all the way back toward the handle 630, meaning the wire 680 at this point is immobile within the body 620 with the plate 660 cinching down/gripping the wire 680 and the nose portion 655 being in its fully retracted configuration in which the portion 655 cannot move any farther back toward the body 620. As also shown in FIG. 6H, the table 695 indicates different wire “pullout forces” that inform maximum compression forces of the springs 290 and 689 so that the resulting spring compression forces are set just below the amount of force that would otherwise be applied at this point of the trigger pull to pull the wire 680 away from the bone/plate 696 during fracture alignment and reduction (with the max compression force of each spring being exerted after the wire has been anchored into the bone/plate 696 via a wire driver). Note that the force values in the table 695 may vary based on insertion depth and bone quality. Also note that the values in the table 695 are to be used for wires inserted to a depth between 10 millimeters (mm) and 20 mm.

As shown in the table 695, for a wire with a diameter of 1.6 mm, a smooth (non-threaded) wire pullout force in Newtons (N) may be between 20 N-75 N for a wide range of bones and, more preferably, 50 N-60 N as optimal for most bones. A partially threaded wire pullout force for a wire with a diameter of 1.6 mm may be between 130 N-225 N (the wire threaded at and through the anchor site) for a wide range of bones and, more preferably, 150 N-200 N as optimal for most bones. For a wire with a diameter of 2.4 mm, a smooth wire pullout force may be between 70 N-130 N for a wide range of bones and, more preferably, 80 N-100 N as optimal for most bones. A partially threaded wire pullout force for a wire with a diameter of 2.4 mm may be between 175 N to 275 N for a wide range of bones and, more preferably, 210 N-250 N as optimal for most bones.

Thus, in one particular non-limiting example as optimal for most bone densities, the compression spring 689 may have a maximum compression force amount of below 50 N for wires having a diameter of 1.6 mm if smooth, and below 150 N if partially threaded. Also in non-limiting examples, for wires of 2.4 mm diameter, the compression spring 689 may have a maximum compression force amount below 80 N for smooth wires and below 210 N for partially-threaded wires.

What's more, in one particular non-limiting example, the reduction handle nose spring 689 maximum force amount may be set below a partially threaded wire pullout value per the table 695 to reduce the risk of partially threaded wire pullout while, in the same implementation, the nose spring 290 maximum force amount may be set below a smooth wire pullout value per the table 695. This may be done for a reason described in detail below in reference to FIGS. 6O and 6P, but may also be done so that, should the martini device 1100 be used without the apparatus 600, the spring 290 for the martini device 1100 may maximize compressive force while reducing the risk of pulling a smooth wire out (as smooth wires are used quite often). But should the physician instead elect to use the apparatus 600 to advance the martini device 1100 over a partially threaded wire (increasing bone purchase at the threads/anchor site interface), the spring 689 may exert more force yet still have that force be below the threaded wire pullout point.

Now suppose that, while the nose portion 655 is in its fully extended position, the physician wants to lock the nose portion 655 in place in this extended position (with respect to the body 620) to establish an overdrive configuration in which the physician can push the nose portion 655 and body 620 forward together without the nose portion 655 retracting toward the body 620 against spring bias as described above. This may advantageously allow the physician to apply as much compressive force along the axis of the wire as desired to push the martini device 1100 forward for secure alignment (e.g., where the patient has healthy bone and risk of wire pullout is therefore less).

To establish this overdrive configuration, with friction between the front end of the portion 655 and the device 1100 existing and/or with another hand holding the portion 655 in place, the physician may use (twist) the handle 630 to radially rotate the body 620 ninety degrees with respect to the portion 655 according to the longitudinal axis 621. This action may cause the aforementioned tabs 685 to themselves rotate radially along the axis 621 and within the body 620 to lock the nose portion 655 in its extended configuration and therefore locking the position of the nose portion 655 with respect to the body 620.

Refer to FIGS. 6I-6L for further understanding. FIG. 6I again shows an exploded partial view of the apparatus 600 and further illuminates the cross-sectional views of FIGS. 6J-6L via its indications of the corresponding views for those figures. Thus, it may be appreciated that FIGS. 6J and 6K are side cross-sectional partial views of the body 620 and portion 655, while FIG. 6L is a top cross-sectional partial view of the body 620 and portion 655. It is to be understood that these figures demonstrate rotation of the tabs 685 of the nose portion 655 within the body 620 to lock the nose portion 655 in overdrive/fully extended configuration as mentioned above.

As shown in FIG. 6J, tab cavities 697 for receiving the tables 685 have been molded into the body 620. The portion 655 has not yet been rotated with respect to the body 620 in FIG. 6J to establish the locked/overdrive configuration and, as such, the tabs 685 have not yet been rotated ninety degrees and into the cavities 697. Also note that the tabs 685 are difficult to see in FIG. 6J owing to the cross-sectional view shown since the tabs 685 are oriented in the viewing plane itself. But once the tabs 685 are in fact rotated ninety degrees as described above, FIG. 6K shows that this rotation ultimately moves the tabs 685 into the cavities 697 as a result. According to this (locked) configuration per FIG. 6K, the tabs 685 cannot travel past, and may even abut, vertical rear walls 698 of the cavities 697 that face forward toward the nose 655. This locks the nose portion 655 in place with respect to the body 620 so that the nose portion 655 cannot travel backward toward the body 620. FIG. 6L also shows this locked configuration in top cross-sectional view.

Then when desired, the physician may rotate the tabs 685 ninety degrees opposite the previous ninety-degree rotation. This moves the tabs out of the cavities 697, placing the nose portion 655 back into the unlocked configuration as shown in FIG. 6J. The tabs 685 may thus again move linearly within the body 620 according to the longitudinal axis 621, which also allows the nose portion 655 itself to move linearly back and forth (against and with spring bias from the spring 689) as the nose portion 655 telescopes back and forth with respect to the body 620.

Also note that in some examples, the tabs may be located on the body 620 and the cavities may be located on the nose portion 655 to still provide a locking mechanism as described above.

The side cross-sectional views of FIGS. 6M-6P further illustrate the locking aspect described above in relation to the martini device 1100 as already positioned against a bone or plate 696. Specifically, FIGS. 6M and 6N show the unlocked configuration where the nose portion 655 can move proximally and distally with respect to the body 620, whereas FIGS. 6O and 6P show the locked/overdrive configuration.

As shown in FIG. 6M, the nose portion 655 is in its fully extended position with respect to the body 620 during provisional bone alignment and fracture reduction, with it being further noted that the trigger 610 is in its fully extended position while the surgical guide wire 680 concurrently extends through the body 620, opening 656, nose portion 655, and martini medical device 1100. Also note that there is no space between the nose portion 655, martini device 1100, and bone/plate 696 linearly along the axis of the wire 680 as the nose portion 655 is touching the rear of the martini device 1100 to push the front of the martini device 1100 up against the bone/plate 696.

FIG. 6N then shows that the portion 655 has been retracted all the way back to its fully collapsed/retracted position due to a subsequent trigger pull (with the trigger pull itself moving the devices 600, 1100 farther forward along the wire 680 toward the bone/reduction site if any space somehow remains between those components and the site 696). As such, additional wire 680 is shown behind the apparatus 600, as previously located within the apparatus 600 prior to that trigger pull. Also note here that both of the springs 689 and 290 concurrently bottom out (are fully retracted) per this configuration, limiting the amount of pull force exerted on the wire 680 due to the trigger pull.

FIG. 6O then demonstrates that, as described above, the physician may grip the handle 630 to rotate the body 620 ninety degrees (or another preconfigured amount) while the nose portion 655 does not concurrently rotate, which in turn rotates the tabs 685 into the cavities 697. Again this locks the portion 655 in place with respect to the body 620. The physician may then use the handle 630 to apply even greater force along the wire 680 and toward the bone/plate 696 to push the martini device 1100 even more tightly against the bone/plate 696, which may be desirable in some instances where bone quality is relatively good and greater compression force is desired across the fracture.

Or, as shown in FIG. 6P, the physician may pull the trigger 610 while the device is in the overdrive/locked configuration and, with the nose portion 655 no longer absorbing any of the resulting action due to it being locked in place, whatever amount of trigger force is being applied by the physician may be translated down the wire 680 to similarly compress the martini device 1100 against the bone/plate 696. To reiterate, that applied force may be greater than would be afforded by the device 600 in the unlocked state via the nose spring 689 and total/maximum travel of the nose portion 655 in the unlocked state, such that in the locked configuration the martini device 1100 may be compressed with greater force against the bone/plate 696 in instances where the wire 680 has good bone purchase on relatively healthy bone.

Additionally, as alluded to in reference to the table 695 above, in one specific non-limiting instance owing to the different maximum force amounts for the springs 290 and 689 as described above, a first trigger pull may be used to advance the martini device 1100 up against the bone/plate 696 with the nose of the martini device 1100 telescoped all the way back (and hence the spring 290 fully compressed), but at the same time the nose portion 655 of the device may not be fully retracted due to the comparatively stronger compression force of the spring 689.

Then on a subsequent trigger pull with the martini device's telescoping nose already retracted, the nose portion 655 of the apparatus 600 may itself retract when not in overdrive to avoid pulling the wire 680 away from the anchor site. Or if the apparatus 600 is placed in overdrive, this subsequent trigger pull may apply even greater force along the wire to further compress the martini device 1100 against the bones and hence compress the fractured bones themselves.

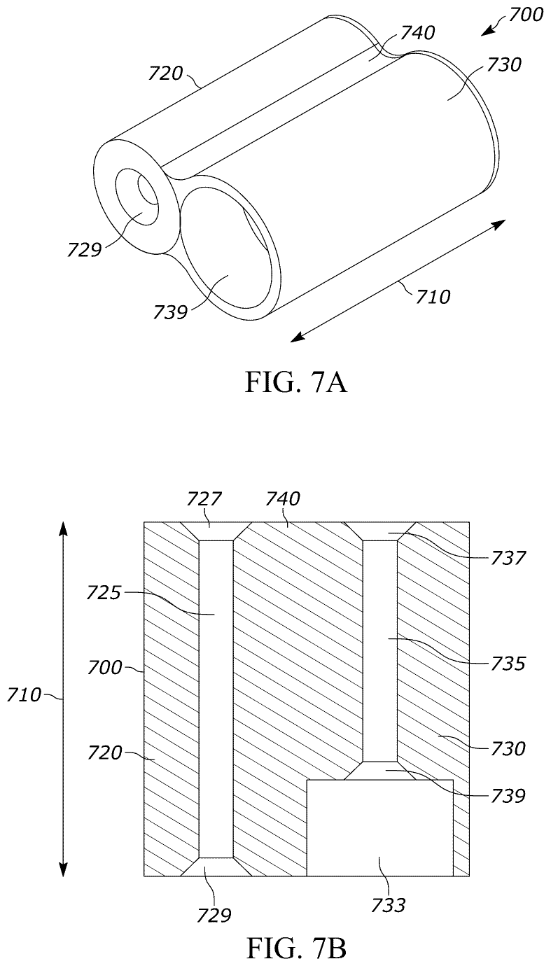

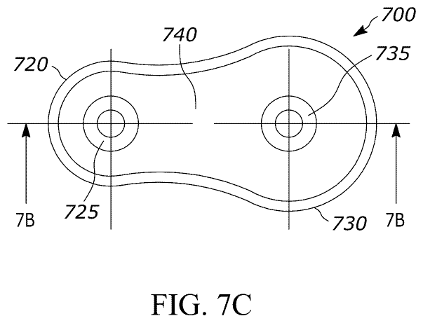

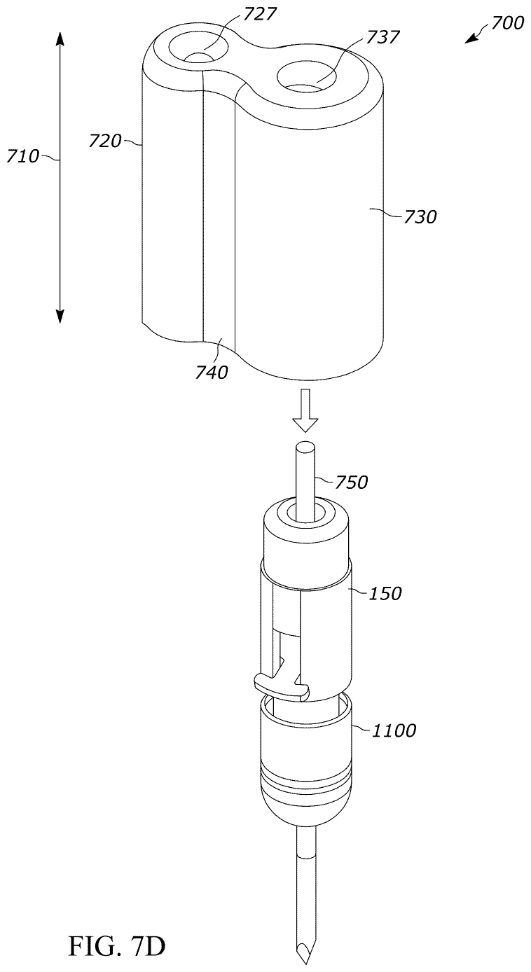

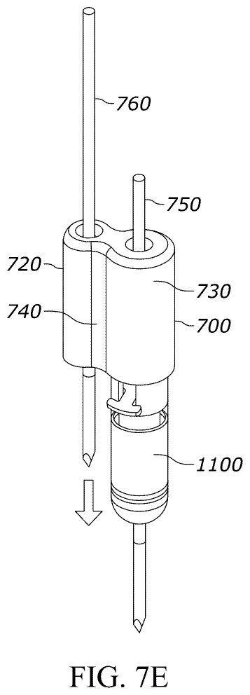

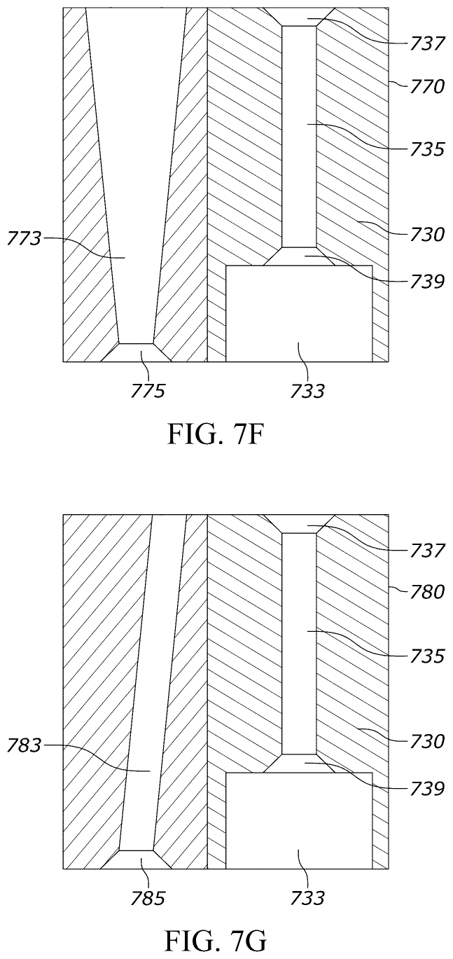

Now in reference to FIGS. 7A-7E, these figures show an example rigid, cannulated guide wire targeting device 700 that may be used consistent with present principles (e.g., used for cannulated screw alignment). It is to be understood in reference to the device 700 that physicians might desire to use cannulated screws and k wires together to pre-align a fracture fragment prior to permanent fixation, and then perform the permanent fixation itself. As such, there may be instances where the physician may wish to align guide wires for two or more canulated screws side by side at a fracture reduction site, and then use those screws for permanent fixation. As part of this technique, the physician might use the device 1100 to compress the fracture, then slide the device 700 down the wire through one tube to meet the device 1100, and then insert and align other wires through other tubes of the device 700.

With this in mind, FIG. 7A shows an isometric view of the device 700, FIG. 7B shows a side cross-sectional view of the device 700, FIG. 7C shows a transverse cross-sectional view of the device 700, and FIGS. 7D and 7E show isometric views of the device 700 as used with the device 1100 during fracture reduction.

Beginning first with FIG. 7A, it may be appreciated that the guide wire targeting device 700 may define a longitudinal axis 710. The device 700 may also define a first hollow, cylindrical tube 720 and a second hollow, cylindrical tube 730 that runs parallel to the first tube 720. The tubes 720, 730 may be coupled to each other by a connector 740 that may be made integral with the tubes 720, 730 in non-limiting embodiments.

As may be appreciated best from FIG. 7B, the first tube 720 may include a cylindrical, hollow opening 725 that extends lengthwise through the tube 720 according to the longitudinal axis 710. The cylindrical opening 725 may have a uniform diameter, and may be bounded at each end by hollow frusto-cone openings 727, 729 that get wider as they extend distally. Note that the openings 727, 729 may provide access from exterior to the device 700 for insertion and passing through of cannulated screws in some instances, as described in greater detail below.

As also shown in FIG. 7B, the second tube 730 may include a cylindrical, hollow opening 735 that extends lengthwise through the tube 730. The opening 735 may also be uniform in diameter, but may be shorter than the opening 725 in the lengthwise dimension. But still, the diameters of the openings 725, 735 may be the same or similar. The opening 735 may also bounded at each end by hollow frusto-conical openings 737, 739, with the opening 737 also providing access from exterior to the device 700. The openings 737, 739 also get wider as they extend distally from the lengthwise center of the device 700. However, distinguishing the first tube 720 from the second tube 730, the lower opening 739 terminates at another cylindrical opening 733 that has a diameter wider than the diameter of the opening 735. In at least some embodiments, the opening 733 may be one or more orders of magnitude larger in diameter than the opening 735. The opening 730 may provide access from exterior to the device 700 to the other hollow components of the tube 730 from the bottom of the tube 730, whereas the opening 737 may do so from the top of the tube 730.

For greater understanding of why the opening 733 is larger in diameter than the opening 735, refer to FIGS. 7D and 7E. During alignment of factures in fracture reduction surgery, the martini device 1100 may first be slid forward along a k-wire 750. Note again that the martini device 1100 per this example includes a lever as its release mechanism rather than the aforementioned slider 160, but in all other respects may be the same as the device 100 as already described above.

The device 700 may then be slid onto the wire 750, and then down to and behind the device 1100 (distal to the fracture site), through the tube 730. In particular, the opening 733 may be positioned proximal to device 1100 while the opening 737 may be positioned distal to the device 1100 when sliding. The wire 750 may therefore be slid through the opening 733, then the opening 739, then the opening 735, and then through the opening 737 for the opening 733 to come down on top of and receive the portion 150 in interference fit.

FIG. 7D therefore demonstrates an appropriate positioning of the device 700 over the device 1100, with the device 700 approaching the anchored end of the k-wire 750. FIG. 7E then shows the device 700 sliding down the wire 750 to meet the device 1100 and engage the device 1100 in the opening 733 in an interference fit. Note that the second end portion 150 (rear end) of the device 1100 may thus be closely received by the opening 733 so that the portion 150 extends at least partially into the opening 733.

FIG. 7E also demonstrates that after this portion of the procedure, a second k-wire 760 may be extended through the tube 720 created by the openings 727, 725, and 729 and down to the fracture site for anchoring at the plate and/or bone. The device 700 may thus stabilize and help maintain, at a fixed distance, alignment and positioning of the wires 750, 760 with respect to each other using the device 1100 so that screws may ultimately be secured at the corresponding anchor locations of the respective wires 750, 760.

The cross-sectional views of FIGS. 7F and 7G then show alternate example embodiments of cannulated screw alignment devices 770, 780 consistent with present principles. In both of these figures, the tube 730 may be the same as described above. But the tube 720 has been replaced with different kinds of tubes.

Specifically, FIG. 7F shows that another tube 773 may be V-shaped, tapering inward form top to bottom for instances where the physician may find that desirable for arranging the guide wire for provisional alignment, adjusting the angle of wire insertion as desired. The bottom of the “V” may then terminate in a frusto-cone opening 775 similar to the frusto-cone opening 729. Yet note that even here the long axis of the tube 733 is still parallel to the long axis of the tube 730.

FIG. 7G then shows another example embodiment of a cannulated screw alignment device 780 where the tube 730 is still configured on the device 780, but a second tube 783 runs obliquely with respect to the tube 730 rather than parallel to it. This may assist the physician in inserting a wire at a defined oblique angle with respect to the first wire where the particular fracture reduction at hand might call for it. Yet note that here too, a frusto-cone opening 785 may be similar to the frusto-cone opening 729.

Thus, it may be appreciated from FIGS. 7F and 7G that the k-wires that get extended through the tube 773 or 783 can still be aligned where a physician wants it using one of these alternate embodiments, with the other k-wire as already extending through the tube 730 advantageously being used as a reference and with the device 1100 helping maintain the alignment and stabilize the arrangement.

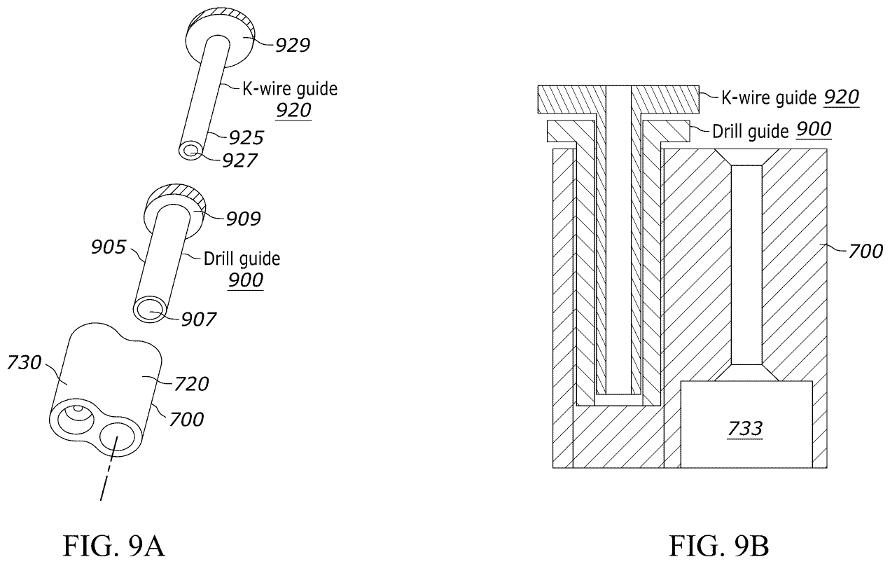

Then after the wires are aligned as desired, the device 700, 770, or 780 may be removed (withdrawn) from the wires. The free ends of the wires may then be fed through cannulated screws, with the screws being slid down to the reduction site. The screws may then be screwed into the bone over the wires and at the wire anchor locations for permanent fixation, at which point the wires themselves may be removed from their anchor points. Or as an alternative where the device 700, 770, or 780 stays in place during screw fixation, the embodiment of FIGS. 9A and 9B may be used instead as will be described in a moment.

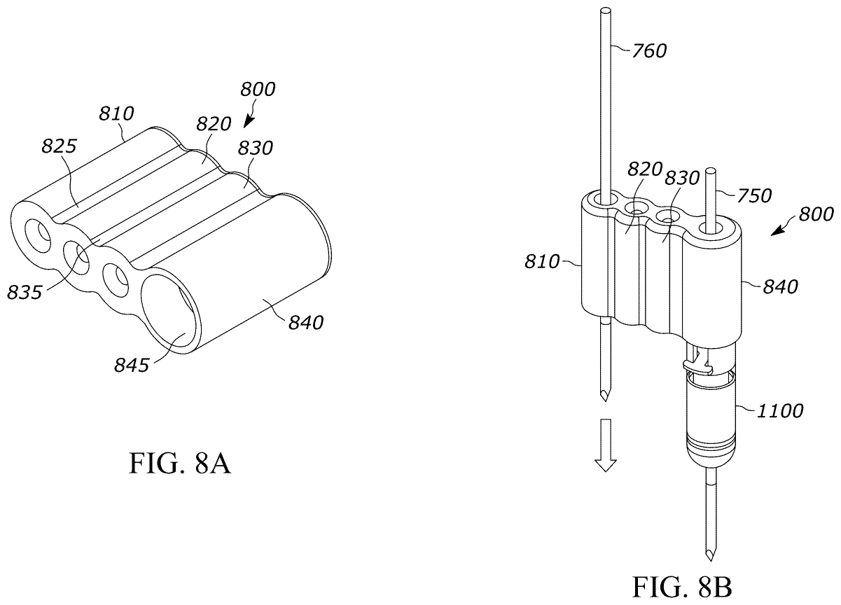

But first, yet another example embodiment of a cannulated screw alignment device 800 will be described in reference to FIGS. 8A and 8B. According to this example, plural tubes 810-830 (three in the present example) may be included in the device, with each connected by a respective connector 815, 825 and made integral with each other along with the connectors 815, 825 themselves. Each tube 810, 820, 830 may be the same as or similar to the tube 720 described above in structure and configuration, or one or more of them may be similar to the tubes 773, 783 in structure and configuration.

But assuming each tube 810, 820, 830 is similar to the tube 720 per one example, note that each tube 810, 820, 830 may define a channel running through the respective tube longitudinally, with each tube being parallel to the others. Also note that although three tubes 810, 820, 830 are shown, more or less but still plural tubes of similar structure (different from the tube 840) may be included on the device 800.

As just mentioned, the device 800 may also include the tube 840, which may be the same as or similar to the tube 730 in structure and configuration. As such, the tube 840 may include an opening 845 similar to the opening 739 to therefore receive the portion 150 of the device 1100 as illustrated in FIG. 8B for stabilization and maintaining wire alignment. The plural tubes 810, 820, 830 therefore also advantageously allow for alignment of multiple screws for fixation at the fracture site at locations beneath each tube 810, 820, 830.

Moving now to the aforementioned FIGS. 9A and 9B, these figures show the device 700 in relation to still other medical device components that may be used consistent with present principles, with it being further noted that the devices 770, 780, and/or 800 may also be used in relation to these other components if desired. In any case, as shown in FIG. 9A, rigid devices 900, 920 may also be used with the device 700. The device 900 may be used as a drill guide for receiving a cannulated drill bit for drilling cannulated surgical screw bores into the fracture reduction site, whereas the device 920 may be used as a k-wire guide for a k-wire.

Therefore, the devices 900, 920 may be nested together in the device 700 as shown in FIG. 9B for the k-wire to be inserted and then anchored. The device 920 may then be removed/withdrawn away from the fracture site for the cannulated drill bit to then be slide over the k-wire and down to the fracture site to drill a hole for a cannulated screw (with the device 920 removed). The drill guide 900 may then be removed/withdrawn so that the larger diameter of the tube 720 subsequently allows the screw head (and rest of the cannulated screw) to be extended through the tube 720 and down to the fracture site for screwing into the hole that was just drilled. All this therefore helps maintain alignment between provisional and permanent fixation for precision.

In light of the foregoing, it is to be understood that each device 900, 920 may include a respective cylindrical lower portion 905, 925 defining a hollow channel on the inside which is accessible from an opening 907, 927 at a respective lower end. The diameters of each channel may be different from each other, and indeed the diameters of the portions 905, 925 may be as well. Specifically, the diameter of the channel defined by the portion 905, and the larger diameter of the portion 905 itself, may be respectively larger than the diameter of the channel defined by the portion 925 and the diameter of the portion 925 itself.

Each device 900, 920 may also include a respective disc or relatively shorter cylinder 909, 929 defining an opening at the top (this opening not shown) that provides access to the respective channel in each device 900, 920. Also note that the channel inside the device 900 may have a diameter that is the same as or slightly larger than the diameter of the drill bit itself, while the channel inside the device 920 may have a (smaller) diameter that is the same as or slightly larger than the k-wire. Also, the smallest diameter inside the portion 720 itself may be the same as, or slightly larger than, the diameter of the head of the cannulated screw.

Again note that FIG. 9B shows the devices 700, 900, and 920 nested together during fracture reduction surgery. It may be appreciated that these components therefore combine to provide a cannulated screw system. And to reiterate, during fracture reduction, the physician may extend the k-wire through the innermost channel defined by the k-wire guide 920 and secure the k-wire to the bone or plate. The physician may then withdrawal the k-wire guide 920 from the k-wire itself (away from the fracture reduction site) and then feed the k-wire through a cannulated drill bit. The bit may then be extended into and through the channel inside the guide 900 for the drill to drill over/around the wire into the bone. The drill guide 900 may then be withdrawn away from the bone site and off the k-wire so that a cannulated surgical screw may then be placed over the k-wire, slid down toward the fracture reduction site, pushed or otherwise extended through the channel in the portion 720 of the device 700, and ultimately screwed into the hole in the bone that was created by the drill bit. The k-wire may then be disengaged from the bone/plate and withdrawn from the patient through the cannulated inner channel of the screw itself and then through the channel in the portion 720. The device 700 main remain stationary throughout this process, acting as an anchor to maintain bone alignment while working on a proximate bone site, because the opening 733 remains engaged the device 1100 (not shown in FIG. 9B) whilst the device 1100 maintains fracture alignment via the separate k-wire that is anchored into the bone adjacent to the screw and compressed by the device 1100 against the bone/plate.

Further note that if the device 800 where used instead of the device 700, this process using the nesting components above may then be repeated to affix additional screws into the bone/plate through the other channels of the other portions 810, 820, 830. The same process might also be used with the devices 770, 780. What's more, in some examples, the devices 700, 770, 780, and 800, as well as the guides 900 and 920, may be provided together in a kit with other components mentioned herein, including the screws, k wires, and drill bits. This may be done so that the physician can pick and choose which components to use and in which combination, depending on the particular fracture reduction at hand.

What's more, note that self-drilling screws may be used in certain instances according to the above and, as such, the aforementioned cannulated drill bit might not be used. Instead, the k-wire may be guided down to the surgical site through the guide 920 while the guides 920 and 900 are nested with each other and the device 700, and then both the guides 920, 900 may be removed from the device 700 to extend the screw through the channel in the portion 720 and down to the surgical site where it may be screwed into the bone and/or plate.

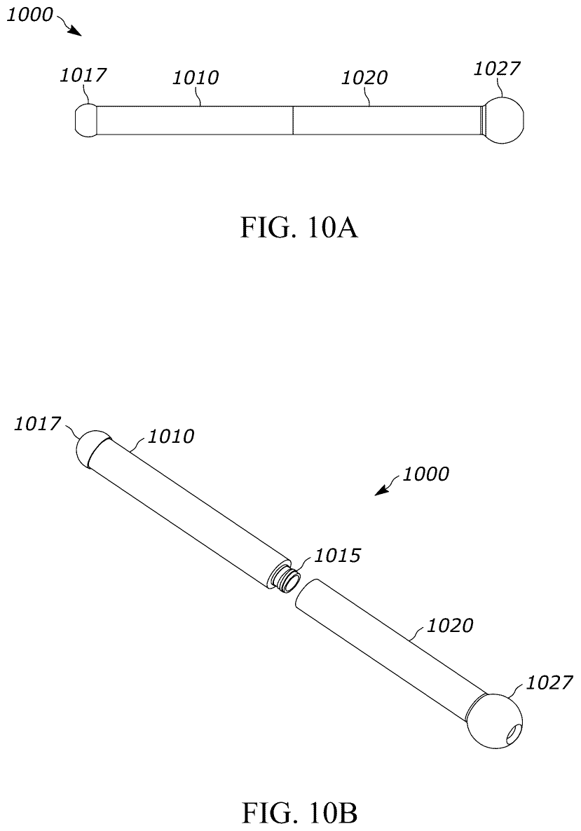

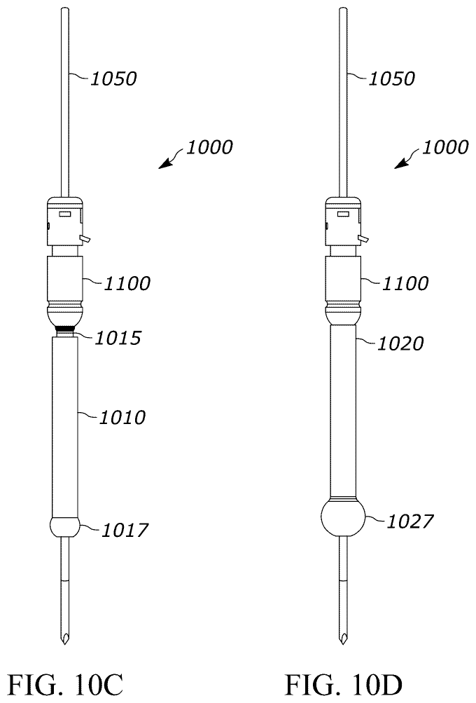

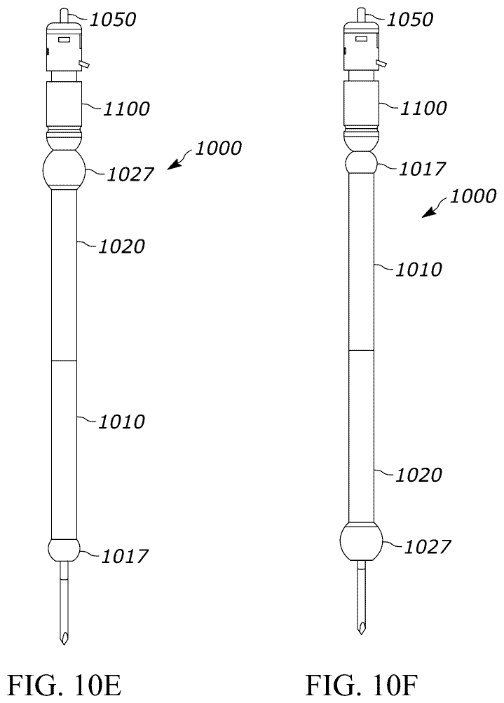

Continuing the detailed description in reference to FIGS. 10A and 10B, these figures show another example rigid, cannulated device 1000 that may be used with the other devices mentioned herein for fracture reduction. Specifically, the device 1000 may be used as an extension rod for provisional fracture alignment using the device 1100, such as instances where fracture reduction is being performed percutaneously through a small hole in the patient's skin and the fracture site is relatively deep inside the patient. The device 1000 may therefore be used to extend into the patient while the device 1100 remains outside of the patient for ease of manipulation by the physician while still enabling and maintaining compression along the k wire (and device 1000) with the device 1100 relatively more out of the way.

This aspect will be discussed in greater detail in reference to FIGS. 10C-10F, but first note that the device 1000 itself may include reciprocal portions 1010, 1020 that may screw together using male threads 1015 on the portion 1010 and female threads (not shown) on the portion 1020 (or vice versa). The portions 1010 and 1020 may be the same overall length, or the portion 1020 may be longer than the portion 1010 (or vice versa). The portions 1010 and 1020 may each define hollow channels extending longitudinally through them as well, with the channels forming a common coaxial channel when the portions 1010, 1020 are screwed together.

Additionally, at a distal end portion of each portion 1010, 1020 may be a respective spherical or semi-spherical ball 1017, 1027. The ball 1027 may have a larger diameter than the ball 1017. Each ball may itself include a distal opening and may be hollow on the inside to also form part of the aforementioned common coaxial channel. Each ball 1017, 1027 may be made integral with or otherwise coupled to its respective portion 1010, 1020. The portions 1010, 1020 and balls 1017, 1027 may rigid and made of surgical-grade metal, plastic, polymer, etc.

The different diameters of the balls 1017, 1027 allow the device 1000 to be variably used in a variety of ways to maintain alignment of bone fractures and a surgical plate using a k-wire 1050. Refer to FIGS. 10C-10F for greater understanding and note that one or both of the portions 1010, 1020 may be used alone or in combination depending on the fracture reduction at hand.

FIG. 10C shows the portion 1010 being used with the device 1100, sans portion 1020. Note that the portion 1010 is proximal to the fracture site compared to the more distally-located device 1100 relative to the patient/fracture reduction site. Yet the device 1100 still helps to maintain the fracture alignment along the k-wire 1050 and prevent movement of the portion 1010 distally away from the reduction site in series with the portion 1010 (via the compression that gets translated through both components). Also note that the portion 1010 may potentially extend into and through the patient's skin and onto the bone, with it being understood that the ball 1017 may be positioned up against the bone or plate inside the patient. The portion 1010 with relatively smaller ball 1017 (smaller in diameter than the ball 1027) may be used to maintain fracture alignment (ball 1017 pressed up against the bone/plate) in instances where the patient has relatively strong bone quality and/or where a relatively small surgical plate is being affixed to the patient and hence the lesser contact surface area afforded by the ball 1017 may be used.

In contrast, were the patient to have relatively poor bone quality and/or were a relatively large surgical plate to be used, and hence the relatively larger contact surface area of the ball 1027 would be desired, FIG. 10D shows that the portion 1020 with larger ball 1027 may be used instead. Here the ball 1027 also abuts the bone or plate to maintain alignment along with k-wire 1050 and provide compression force in series with the device 1100 (with the device 1100 secured and/or abutting the portion 1020 to still prevent movement 1020 distally away from the fracture site).

FIG. 10E then shows another example where both of the portions 1010, 1020 may be used together (as screwed together per the male/female threads described above) for a combined overall length that is longer, as might also be desired by the physician depending on the nature of the fracture reduction at hand. But similar to FIG. 10C, the ball 1017 may be positioned proximate to/within the patient for circumstances as outlined about with respect to FIG. 10A. FIG. 10F then shows the combined overall length of the portions 1010, 1020 again being used, but with the ball 1027 positioned proximate to/within the patient for circumstances as outlined above with respect to FIG. 10B.

Before moving on to other figures, further note that the device 1000 and/or individual components thereof may be packaged together in a kit within any/all other devices and components discussed herein. Also before moving on, further note that the exterior surfaces of the portions 1010, 1020 may themselves be threaded in certain non-limiting instances (e.g., male or female threads).

The martini device 1100 that has been repeatedly mentioned above will now be described in detail in reference to FIGS. 11A-11C, which show the martini device 1100 with its lever release mechanism. But it is noted that the martini device 1100 may be the same as the martini device 100 in all other respects notwithstanding the swapping of release mechanisms. FIG. 11A is a front isometric view, FIG. 11B is a rear isometric view, and FIG. 11C is a cross-sectional view.

Specifically, rather than a slider release mechanism 160 as described above, the release mechanism per this example implementation may include a lever/tab 1110 coupled to the plate 250. For example, the lever 1110 may be made integral with the plate 250 and, as such, may be made of the same material as the plate itself. The lever 1110 may be manipulable to move the plate 250 about the fulcrum 300 to counteract the spring bias from the spring 230 and permit withdrawal of the surgical guide wire 115 from the third aperture 260 through the first aperture 120. As shown, the distal top portion of the lever may be circular, though other shapes may also be used. Thus, a physician may pull the lever back away from the nose cap 210 according to the longitudinal axis 110 to release the binding action the aperture 260 creates on the wire 115 by aligning the third plane of the plate 250 closer to vertical/perpendicular to the longitudinal axis 110, freeing up the wire 115 to withdraw the device 1100 from the wire 115.

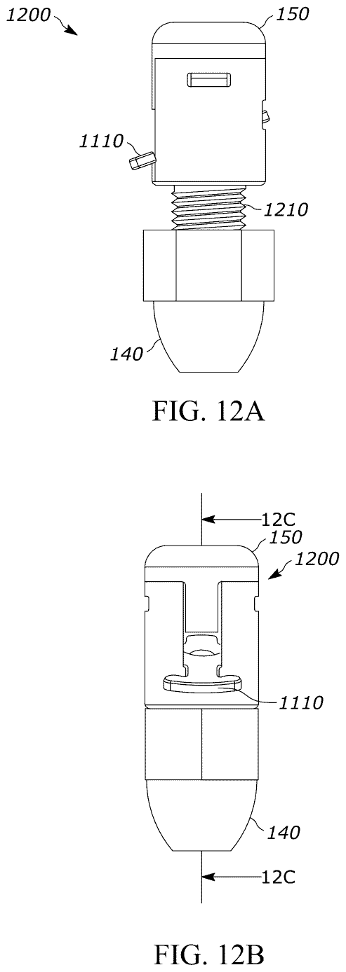

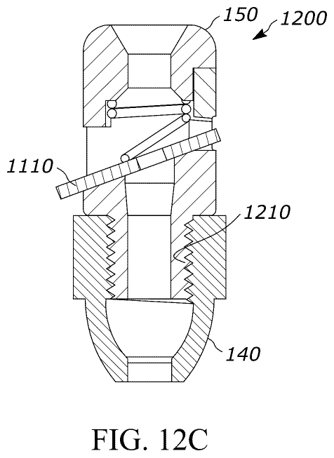

Continuing the detailed description in reference to FIGS. 12A-12C, these figures show another example martini device 1200. FIG. 12A is a side orthogonal view, FIG. 12B is a top orthogonal view, and FIG. 12C is a cross-sectional side view. The device 1200 per these figures may be the same as the device 1100 described above, save for the following differences. Rather than telescoping members that slide back and forth together at the front of the device as described above in reference to FIGS. 1A-1G, here the front end portion 140 may move with respect to the rear end portion 150 via a screw mechanism.

Specifically, the portion 150 may have a post 1210 with male screw threads that engage female screw threads in an interior, reciprocal cavity of the portion 140 as best shown in FIG. 12C. However, further note that in other examples the portion 140 may have the post with male screws and the portion 150 may have the cavity with female screws. In either case, the portion 140 may be screwed/rotated radially in one direction to extend the portion 140 away from the portion 150 as desired by the physician per FIG. 12A (e.g., to increase compression force when the nose of the portion 140 is positioned up against the plate or bone), and screwed/rotated radially in the opposite direction to retract the portion 140 toward the portion 150 as desired by the physician per FIGS. 12B and 12C (to reduce the compression force).

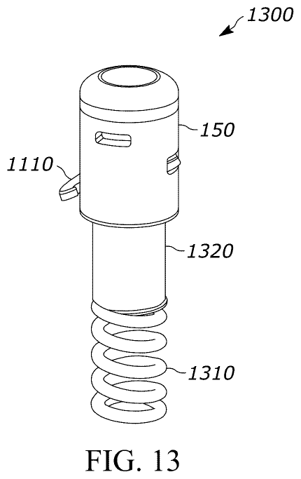

FIG. 13 shows yet another example embodiment of a martini device consistent with present principles, with the martini device 1300 of FIG. 13 being similar to the martini device 1100 as described above save for the following differences. Specifically, here the front portions (e.g., telescoping nose) of the martini device have been removed and replaced with a spring 1310 that is over-molded onto a post 1320. Also note that the cam plate 250 and lever 1100 are located in the rear portion 150. Even if not over-molded, the spring 1310 may nonetheless be made integral with the post 1320 of the housing/body for the rest of the device 1300 (including portion 150). The spring 1300 may be a compression spring in non-limiting examples. Also note that the post 1320 may be cylindrical in shape and have a smaller diameter than the portion 150. Thus, the spring 1310 may still perform as the spring 290 but without the nose portion.

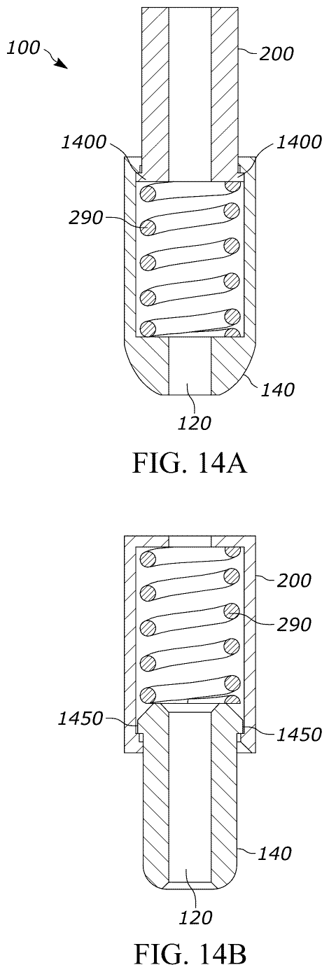

Now in reference to FIGS. 14A and 14B, these figures are partial cross-sectional views that illustrate alternate non-limiting embodiments of the telescoping nose portion of the martini device 100 (and/or martini device 1100) as described above. Specifically, while FIG. 14A shows the nose portion 140 sliding over top of the body 200 (with the nose portion 140 having a larger diameter than the body 200 to do so), FIG. 14B illustrates that the nose portion 140 might instead be configured with a smaller diameter than the body 200 while still allowing the telescoping of those components with respect to each other and the spring action described above to reduce backlash.

In reference to FIG. 14A in particular, note that a ring or rib 1400 similar to mentioned above with respect to FIG. 1A et. seq. may be located on the body 200 (e.g., circumscribing the body 200) and abut inner walls on the nose portion 140 to prevent the nose portion 140 from sliding off of the body 200. In contrast, per FIG. 14B, a ring or rub 1450 may be located on the nose portion 140 and abut inner walls on the body 200 to still prevent the nose portion 140 from sliding off the body 200.

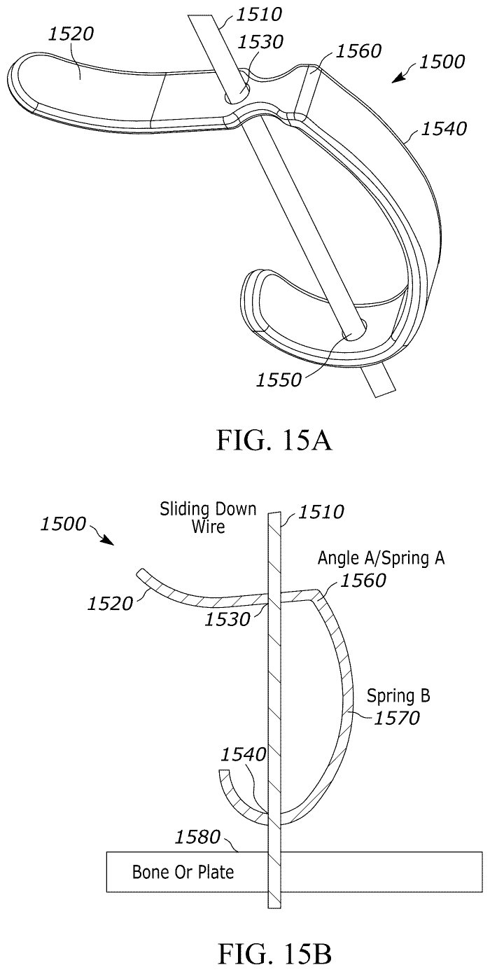

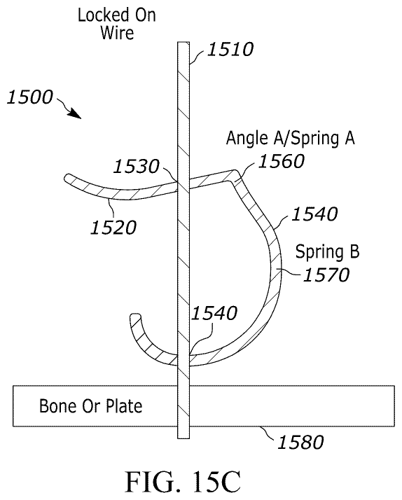

Moving on to FIGS. 15A-15C, these figures show another martini device 1500 that may be used consistent with present principles. Accordingly, as with other martini device examples discussed above, the device 1500 may be advanced down a guide wire 1510 during provisional fracture reduction to apply a compression force at the fracture site. FIG. 15A shows an isometric view of the device 1500, while FIGS. 15B and 15C show a side view of the device 1500 in different device states/configurations. Each aspect of the device 1500 may be made integral with other aspects of the device 1500, with the device 1500 being generally rigid save for spring aspects as described below. The device 1500 may be made of any suitable surgical-grade material, including surgical-grade metal or plastic.

As shown in these figures, the device 1500 may include a handle 1520 with a first aperture/opening 1530 through which the wire 1510 may be fed. The device 1500 may also include a body 1540 with a second aperture/opening 1550 through which the wire 1510 may also be fed. Additionally, a joint 1560 between the handle 1520 and body 1530 may be materially biased to act as a first compression spring, while the body 1540 may be bowed outward convexly, exhibiting material bias to establish a second compression spring 1570 at least at the apex of the bow.

Accordingly, spring 1560 allows for Angle A (relative to the wire 1510) to increase (become closer to orthogonal) so that the device 1500 can slide down the wire 1510. Then when device advancement is obstructed by a plate or bone 1580 as shown in FIGS. 15B and 15C, the continued applied (increasing) force from the physician's hand pressing down on the upper surface (e.g., handle 1520) causes the spring 1570 to activate such that the overall height of the device 1500 decreases as spring 1570 continues to flex (as illustrated in FIG. 15C), Then when the advancement force from the physician is removed, the force from the spring 1570 transfers into the engagement hole 1530 between the wire 1510 as spring 1560 force Angle A decreases, locking the device 1500 to the wire 1510 at the top interface (e.g., as established between the handle portions forming the hole 1530 and the wire 1500 itself). Because spring 1570 is held in a compressed position as shown in FIG. 15C, a compressive force is maintained on the bone/plate 1580.

Thus, a physician may advance the device 1500 down the surgical wire 1510, with each downward press of the handle 1520 region (e.g., the top region of 1500) toward the bone or plate 1580 being used to advance the device 1500 farther down the wire. Since the bottom of the device 1500 has not yet met the bone/plate interface, the device 1500 may slide freely until the bottom of the device 1500 meets the bone/plate interface. Then once the bottom meets the bone/plate, additional downward force acts to compress spring 1540 increasing the force applied to the bone/plate interface through spring 1540. Once the surgeon releases his/her manual force from device 1500 as applied through the aforementioned additional advancement, the compression spring 1570 pushes against the bone/plate interface and translates through spring 1560, resulting in an increasing moment about the contact points between the wire 1510 and the through hole 1530 to lock the martini device 1500 on the wire 115 (e.g., such that the martini device 1500 cannot move backward any additional distance relative to wire 1510 due to the handle 1520 becoming more obliquely oriented cinching on wire 1520 at through hole 1530 (i.e. wire/through-hole interface)). The spring 1570 force is then maintained between the wire/through-hole interface and the front of the device 1500 which is still obstructed by the plate/bone. This, in turn, results in device 1500 using the anchored wire 1510 to apply a compressive force through spring 1540 to the bone/implant and even through the aligned (fractured) bones.

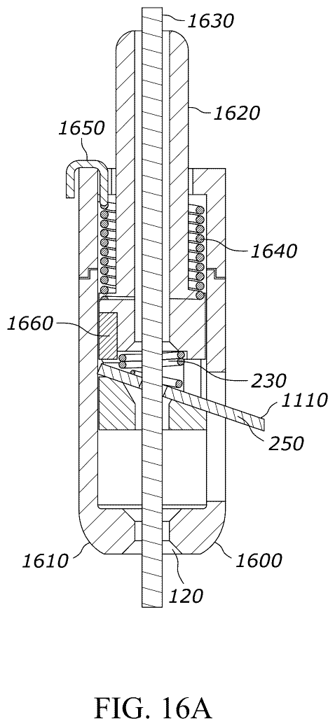

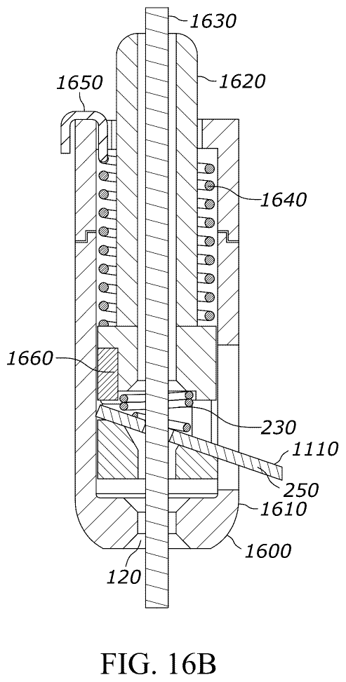

Continuing the detailed description in reference to FIGS. 16A and 16B, these figures shows another example martini device like the martini device 1100 but with some components located within the nose portion 1610 rather than separately in the body 1620 apart from the nose portion 1610. And here, further note that the nose spring 1640 is behind the camming components that lock/unlock on the wire (elements 230, 250, and 1110).

Accordingly, the cam plate 250 with lever 1100 and spring 230 may telescope within the nose portion 1610 as coupled to the body 1620 (with the distal portions of the body 1620 themselves telescoping within the nose portion 1610). But both the nose portion 1610 and body 1620 still define the channel inside the device 1600 through which a k-wire 1630 may extend. Also in contrast to the martini devices 100 and 1100, the compression spring 290 has been replaced with an extension spring 1640 that hooks a first U-shaped end 1650 thereof around the proximal end of the nose portion 1610. The spring 1640 may also be over-molded at its opposite end to a plunger portion 1660 of the body, with the plunger portion 1660 including the spring 230 and cam plate 250.

Thus, FIG. 16A shows the device 1600 in a retracted configuration in which the nose portion 1610 is more proximal to the body 1620 under bias from the extension spring 1640 that pulls the two components together, whereas FIG. 16B shows the device 1600 in an extended configuration where the portion 1610 is extended distally away from the body 1620 while a physician slides the device 1600 down the k-wire 1630 (overcoming the spring bias of the extension spring 1640). Upon release by the physician, the extension spring 1640 then retracts the nose portion 1610 toward the body 1620 to absorb backlash.

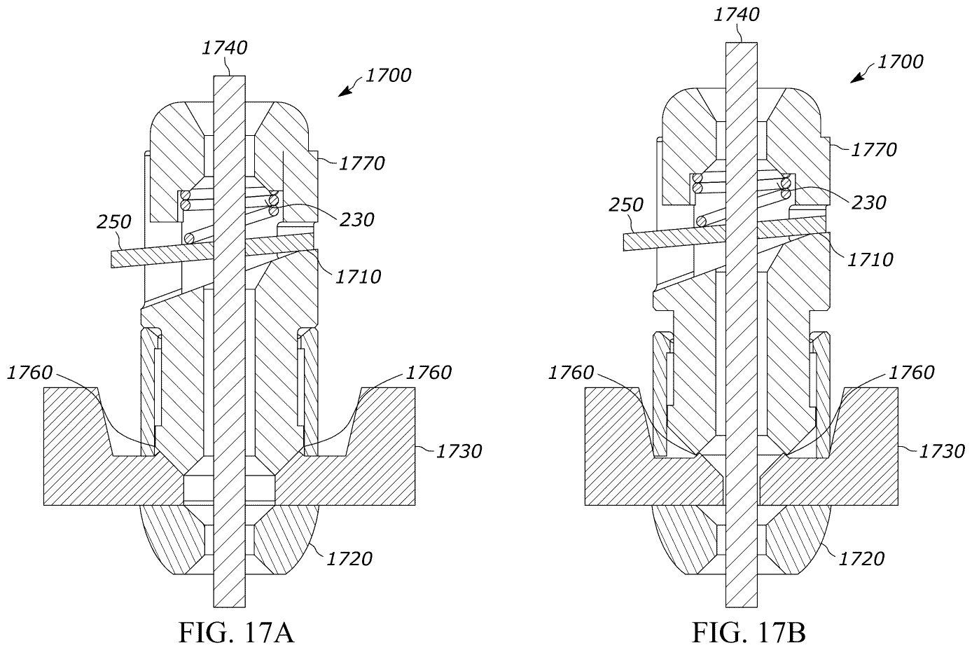

Continuing the detailed description in reference to FIGS. 17A and 17B, these figures show yet another example embodiment of a martini device 1700 that locks onto a guide wire via a cam plate 250 as described herein. Here the plate 250 rests against a fulcrum 1710, with the compression spring 230 biasing the plate 250 to an oblique orientation similar to as described above for other example martini devices as shown in FIG. 17A.

But in contrast to other martini devices mentioned above, here a nose portion 1720 may have wedges 1730 that move transversely inward when pinched (the pinched configuration being shown in FIG. 17B). Thus, the physician might slide the device 1700 down the guide wire 1740 and to the fracture site, but there might still be little to no compression across the fracture site at that point (FIG. 17A). The physician may then pinch the wedges 1730 to push surfaces 1760 of the body 1770, and hence the body 1770 itself, away from the fracture site while the cam plate 250 still bites down on the wire 1740 at the same location, thus increasing the compression force the device 1700 provides at the distal end to the fracture site itself.

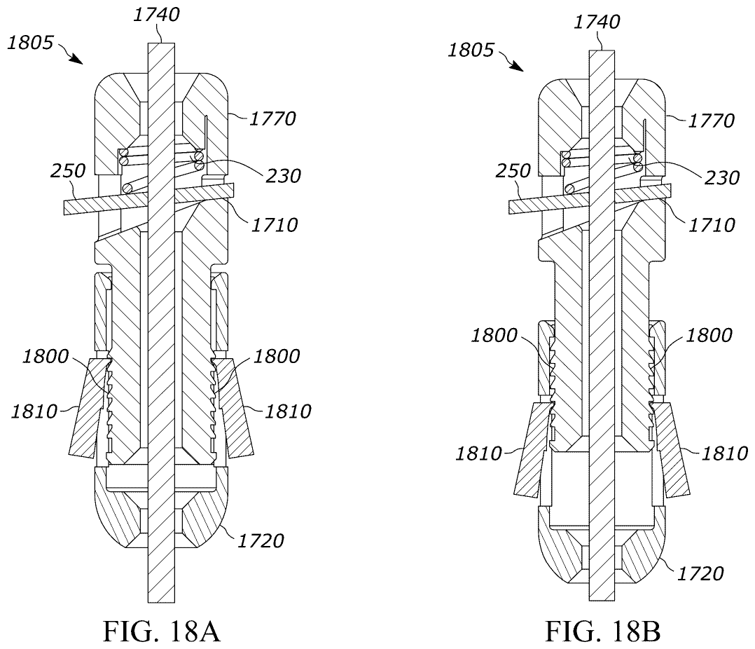

Other mechanical arrangements may also be used in lieu of the wedges to accomplish the same thing. As one example, refer to the martini device 1805 shown in FIGS. 18A and 18B. Here, a ratchet mechanism may be used, where the body 1770 may be pulled away from the nose portion 1720. As a result, teeth 1800 on the body 1770 may be ratcheted past teeth on the tabs 1810 (the tabs being located on the nose portion 1720) so that the teeth on the tabs 1810 then lock into the teeth 1800 on the body 1770 and hold the body 1770 farther and farther away from the nose portion 1720 as the components 1720, 1770 are separated, increasing the compression force. Distal end portions of the tabs 1810 might then be pressed inward to release the compression force if desired.

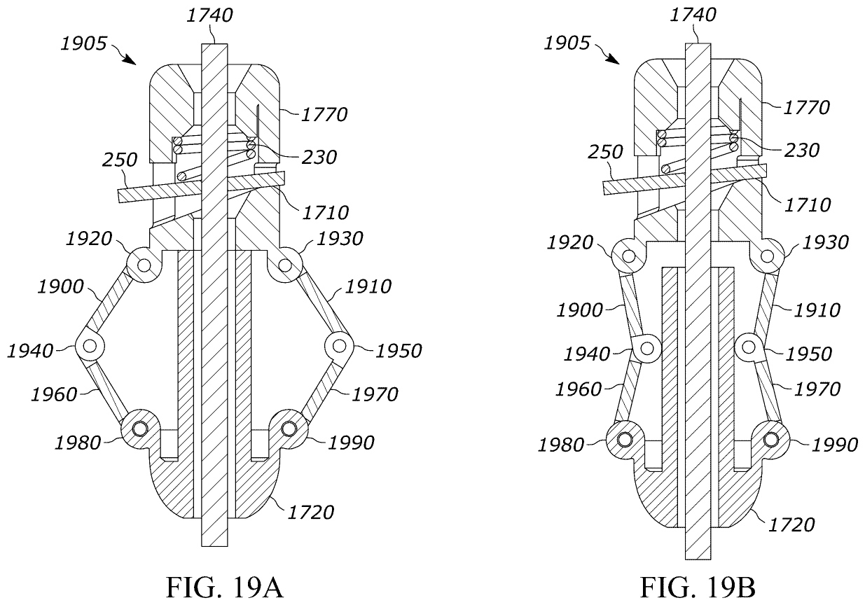

FIGS. 19A and 19B show yet another example martini device 1905. Here, respective upper arm sections 1900, 1910 may respectively rotate about first joints 1920, 1930 and second joints 1940, 1950. The first joints 1920, 1930 couple the upper arm portions 1900, 1910 to the body 1770. The second joints 1940, 1950 are also coupled to respective lower arm sections 1960, 1970, with the lower arm sections 1960, 1970 also coupled to the nose portion 1720 at joints 1980, 1990. Thus, the physician may advance the martini device 1905 down to the fracture site with the arm portions bowed out as shown in FIG. 19A. The physician may then press inwards at or around the joints 1940, 1950 to collapse the arm portions inward as shown in FIG. 19B to increase the compression force.

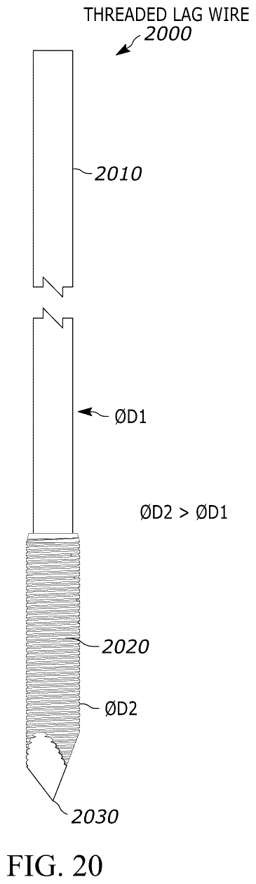

Moving on to FIG. 20, this figure shows an innovative partially-threaded surgical guide wire 2000 that may be used according to the description above. As shown in FIG. 20, the wire 2000 may include a first segment 2010 that may be cut by a physician to a desired length. The segment has a first diameter that is less than a second diameter of a threaded second segment 2020 of the wire 2000. Also note that the segment 2020 has a pointed distal tip 2030.

Thus, the threads on the segment 2020 may be extended through provisional holes in the patient's bone to capture a far piece of the bones and/or plates that are being aligned together. The threads and larger diameter of the segment 2020 may thus help create compression force on one end of the aligned bones/plates, while the devices 100/600 (or 1100/600) create compression force on the other end of the aligned bones/plates. In some examples, ridges that circumscribe the outside of the segment 2020 in respective planes perpendicular to the longitudinal axis of the wire 2000 may be used in lieu of screw-type threads that extend down the segment 2020 for even greater bone purchase/engagement.

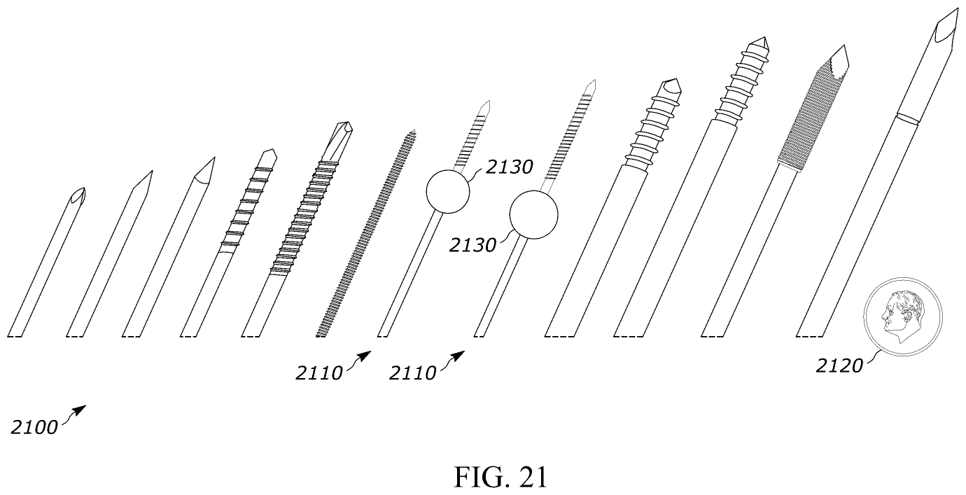

Turning to FIG. 21, additional guide wire examples 2100 are shown that may be used consistent with present principles. Among these guide wire examples are olive wires 2110. The diameters of these flexible wires 2100 (equivalently, rigid guide pins) may be appreciated relative to the dime 2120 shown. Also note that some of the wires 2100 may be threaded while others are not. Also note that the olive wires 2110 may be used, where the olives 2130 may provide compression force at one end of the bone alignment while the device 100 or 1100 may provide compression force at the other end of the bone alignment.

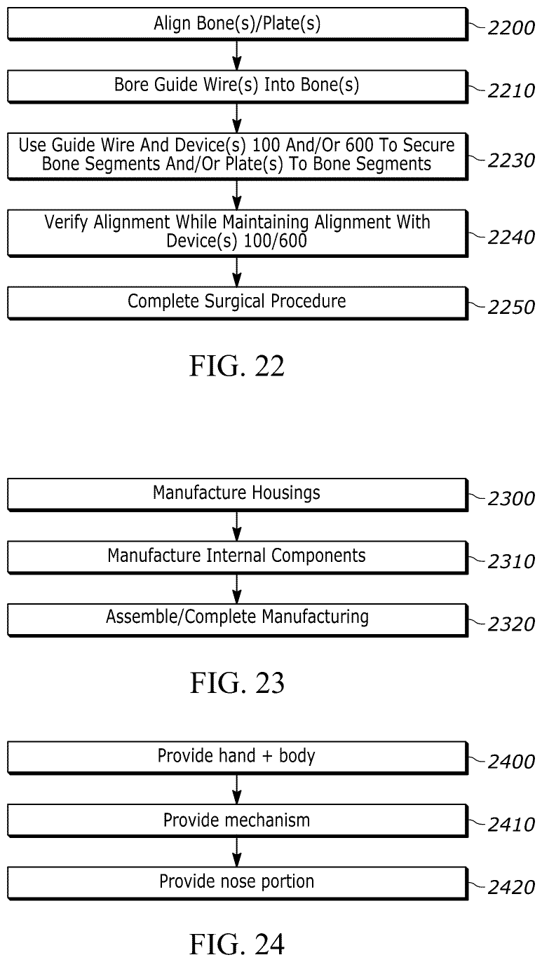

Now in reference to FIG. 22, an example method is demonstrated in flow chart format for surgically using a medical device (such as the device 100 or 1100) consistent with present principles. Beginning at step 2200, the method includes initially aligning various patient bones and/or surgical plates for fixation together consistent with present principles. The method then moves to step 2210 where guide wire(s) (provisional) are bored through the bone(s) according to the desired alignment.

Then at step 2230, the guide wire and the devices 100/600 may be used to provide compression force to secure the bone segments and/or to secure one or more plates to the bone segments, maintaining the alignment. For example, a free end of the wire may be fed through the first aperture 120, then through the aperture 260, and then through the aperture 130. The device 100 may then continue to be advanced/slid along the wire as desired using the apparatus 600 (or by hand) until the device 100 is compressed against one side of the aligned bone structure. Yet owing to the oblique angle of the aperture 260 relative to the longitudinal axis of the device 100 as described above, the device 100 cannot be withdrawn or unintentionally slide off the opposite way along the wire save for using one of the release mechanisms described above (e.g., slider 160 and/or lever 1110).

Then at step 2240 the physician may verify the intended alignment of the bones and/or plates with the devices 100/600 holding the alignment in place (e.g., on one side of the aligned bones while the segment 1220 of the wire 1200 helps maintain the alignment on the other side of the aligned bones/plates). The process may then flow to step 2250 where the devices 100/600 and/or guide wire may be removed from the patient. Also at step 2250, the surgical procedure may be completed with bone/plate alignment verified by drilling permanent holes in the bone(s) and performing permanent fracture reduction. This last step might occur, for example, after one or more alignment adjustments are performed as desired, using the devices 100/600 in the process.

It may thus be appreciated according to FIG. 22 that the medical device and wire may be used to maintain alignment of a first bone with another object (e.g., second bone and/or plate) during the surgical procedure.

FIG. 23 shows an example method in flow chart format for manufacturing some or all of the medical device components described above, such as components the devices 100 and 1100 as well as the apparatus 600, consistent with present principles. Beginning at step 2300, the housing(s) may be manufactured, such as through injection molding, three-dimensional (3D) printing, computer numerical control (CNC) manufacturing, and/or other methods. Thereafter, step 2310 may be performed where the internal components of the device(s) may be manufactured using similar methods. Then at step 2320 manufacturing may be completed, such as through assembling all the parts together for shipping, vending, providing, etc.

Now in reference to FIG. 24, this figure shows an example method in flow chart for providing a medical device and/or hand-held advancement apparatus consistent with present principles. Thus, note that the process flow of FIG. 24 may be used for vending or otherwise providing the medical device and/or apparatuses through the channels of commerce and ultimately to a medical professional.

Thus, the method includes, at step 2400, providing a handle and a body coupled to the handle for the device 600 described above. Then at step 2410 the method may include providing a mechanism coupled to the body, with the mechanism including an arm that is actuatable to move a martini medical device located in front of the apparatus 600 along a surgical guide wire. Then at step 2420 the method may include providing a nose portion coupled to the body of the apparatus 600. The nose portion may extend distally away from the body and retract toward the body against spring bias when the arm is actuated to concurrently move the body of the apparatus 600 and the martini medical device along the surgical guide wire while the martini medical device is obstructed by a surface in front of the martini medical device.

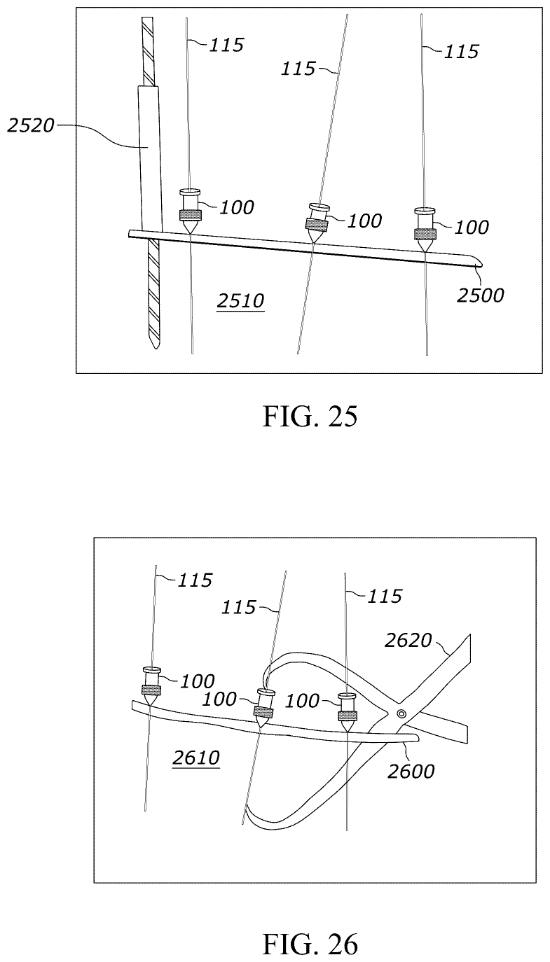

Continuing the detailed description in reference to FIGS. 25-28, these figures show example use cases for the devices 100/600 consistent with present principles to create axial pressure on bones and/or plates along the axis of the surgical guide wire itself.

Beginning first with FIG. 25, respective devices 100 are shown as advanced along respective wires 115 to compress against a surgical plate 2500 that itself is positioned up against a patient's bone 2510. If desired, a surgical tool 2520 may also be positioned against the plate 2500 to help maintain alignment. Thus, according to this example the devices 100 may provide a relatively slight one-sided axial reduction force, no additional surgical clamp being used, once slid down the wire 115 to keep the plate from moving during installation/permanent fixation.

FIG. 26 shows another example. Here, respective devices 100 are again shown as advanced along respective wires 115 to compress against a surgical plate 2600 that itself is positioned up against a patient's bone 2610. A surgical clamp 2620 is also used to compress a respective device 100 on one side of the aligned bone(s)/plate with the other side of the aligned bone(s)/plate. This provides relatively significant axial reduction force to reduce the plate 2600 to the bone 2610 with the clamp 2620.

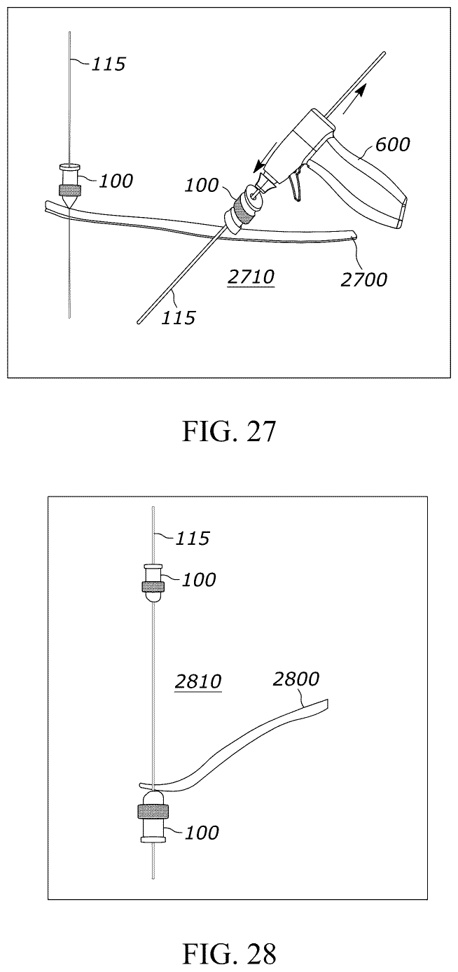

Turning to FIG. 27, yet another example is shown. Here, respective devices 100 are again shown as advanced along respective wires 115 to compress against a surgical plate 2700 that itself is positioned up against a patient's bone 2710. Note that one device 100 is shown being used by itself, while another device 100 is shown as advanced along the respective guide wire 115 using the apparatus 600 before the apparatus 600 is withdrawn from the wire 115 (leaving the device 100 advanced by the apparatus 600 in place and locked along the wire 115 for compression against the plate 2700). This example therefore demonstrates a higher one-sided axial reduction force (no clamp being used) to reduce the plate 2700 to the bone 2710, and/or to capture and temporarily reduce a bone fragment through the plate 2700.

FIG. 28 shows still another example. Here, respective devices 100 are shown facing each other as advanced from opposing sides of a same wire 115, with one of the devices 100 advanced to compress a plate 2800 to one side of a bone structure 2810 and the other device 100 advanced on the other side of the bone structure to compress against an external portion of the structure 2810 itself. This allows for relatively significant axial reduction force to reduce the plate 2800 to the bone(s) 2810 with two sliding pill/tack devices 100.

Moving on from FIG. 28, note that in some specific examples a kit including one or more of the devices/apparatuses disclosed above (and/or sub-components of those devices) may be manufactured, vended, provided, and/or used during a fracture reduction procedure or other type of surgical procedure consistent with present principles. Surgical alignment wires of one or more types disclosed herein may also be provided as part of the kit. Other wires may also be included. The surgeon may thus decide on the fly which wire/device combination from the kit to use, depending on whatever circumstances the surgeon might encounter during surgery.

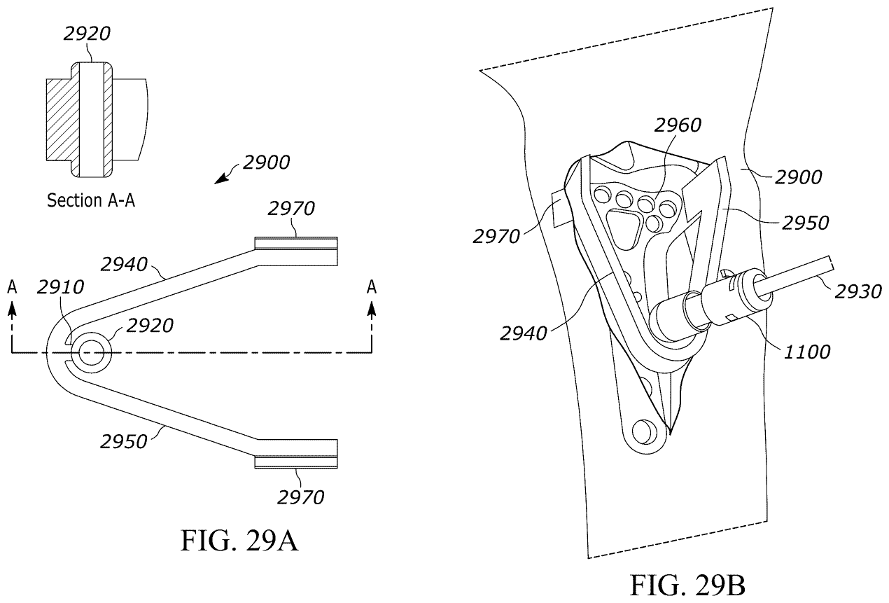

Continuing the detailed description in reference to FIGS. 29A and 29B, these figures show how a martini medical device may be used in combination with a rigid tissue retractor 2900 to spread a patient's tissue apart for a physician to access and visualize a fracture reduction site. As shown in FIG. 29A, the retractor may be generally V-shaped. At the apex of the V, extending inwards along an arm 2910, may be a cylinder 2920. As also shown, the rigid retractor 2900 may resist inward compression at arms 2940, 2950 that extend away from the apex in a V-shape in the X-Y plane to spread tissue. Further note that tabs 2970 may also be located on each arm 2940, 2950 to further aid with tissue retraction (e.g., sliding under the skin in a plane parallel to the retraction plane).

Additionally, it is to be understood that the whole device 2900 may be moveable in the Z-dimension to enable compression with the martini device 1100 using a guide wire 2930 consistent with the disclosure above, while being radially movable in the X-Y plane about an axis of rotation at the center of the cylinder 2920 that is created by the martini device compression. This may aid the physician with visualizing the surgery site and arranging components where desired for fracture reduction.

FIG. 29B therefore shows the retractor 2900 retracting patient tissue to provide access and vision to a plate 2960. The guide wire 2930 may be anchored beneath the plate 2960 to the patient's bone, and also extend through the hollow interior of the cylinder 2920. The martini device 1100 may then come down on top of the upper portion of the cylinder 2920 to compress down on the cylinder 2920, which itself compresses the device 2900 down on the plate 2960 during provisional reduction. Or the martini device 1100 may slide within the cylinder to still provide compression on the plate 2960.

Accordingly, it may be appreciated that the martini device 1100 may provide compression force across the fracture site while also holding the retractor 2900 in place, as well as holding the implant 2960 in place provisionally. The physician may then advantageously adjust the positioning of the plate 2960 within the patient to where the physician wants the plate 2960 prior to permanent fixation to the patient.

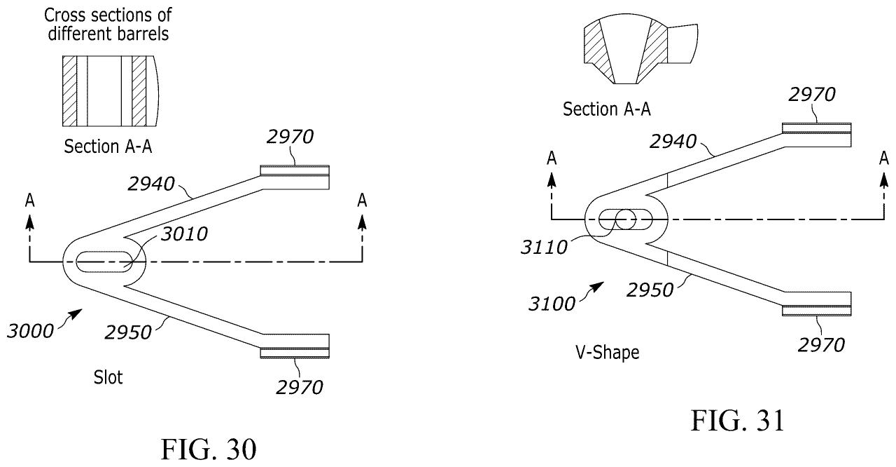

FIG. 30 shows another example rigid tissue retractor 3000 that may be used consistent with present principles. The retractor 3000 may be similar to the retractor 2900 except rather than having a cylinder coming off of the arm 2910, the inside of the apex of the V forms a slot 3010 of uniform depth as illustrated by Section A-A of FIG. 30. So in addition to movements similar to those of the device 2900 as described above, the slot 3010 also enables linear movement of the device 3000 before and during compression along the longitudinal axis created by the slot 3010.

FIG. 31 also shows an example rigid tissue retractor 3100 that may be used consistent with present principles. The retractor 3100 may be similar to the retractor 3000 except that rather than the slot 3010, the retractor 3100 may itself have a V-shaped opening 3110 in the Z dimension such that the opening 3110 tapers/narrows inward as it extends downward. This may be appreciated from Section A-A of FIG. 31. It may therefore be appreciated that the V-shape of the opening 3110 allows Z-dimension angular adjustment of the device 2950 relative to the patient during compression.

Thus, it may be appreciated that the device 2900 allows radially movement of the device 2900 in the X-Y plane, the device 3000 allows radial plus linear movement in the X-Y plane, and the device 3100 allows radial plus angular up/down motion relative to the compression point (e.g., for angulation change with the distal portions of the arms 2940, 2950 changing elevation more than proximal portions).

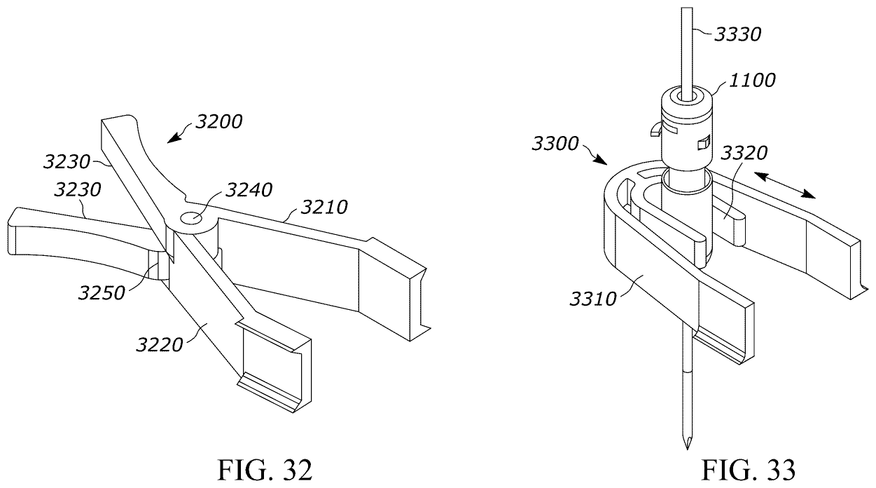

FIG. 32 shows yet another example rigid tissue retractor 3200 that may be used consistent with present principles. Here, retracting arms 3210, 3220 may be spread apart by pressing inwards on scissor handles 3230. The guide wire may extend through a cylinder 3240 at the rotatable junction 3250 of the arms 3210, 3220, as may a martini device to provide desired compression across the fracture site.

FIG. 33 shows still another example rigid tissue retractor 3300 that may be used consistent with present principles. Here, a double-U shape may be implemented, where the outer “U” 3310 may be used to retract patient tissue while. The inner “U” 3320 may first have a guide wire 3330 extended therethrough, and then a martini device 1100 may be slid into the inside of the inner “U” to engage the inner “U” in an interference fit where the inner “U” pinches inwards on the martini device 1100 to stabilize it while the martini device 1100 provides compression across the fracture site.

Components included in one embodiment can be used in other embodiments in any appropriate combination. For example, any of the various components described herein and/or depicted in the Figures may be combined, interchanged, or excluded from other embodiments. “A system having at least one of A, B, and C” (likewise “a system having at least one of A, B, or C” and “a system having at least one of A, B, C”) includes systems that have A alone, B alone, C alone, A and B together, A and C together, B and C together, and/or A, B, and C together.

The term “a” or “an” in reference to an entity refers to one or more of that entity. As such, the terms “a” or “an”, “one or more”, and “at least one” can be used interchangeably herein.

It is to be understood that whilst present principals have been described with reference to some example embodiments, these are not intended to be limiting, and that various alternative arrangements may be used to implement the subject matter claimed herein. Accordingly, while particular techniques and devices are herein shown and described in detail, it is to be understood that the subject matter which is encompassed by the present application is limited only by the claims.

What is claimed is:

1. A hand-held medical device advancement apparatus, comprising:
a housing defining a handle and an elongated body intersecting the handle;
a mechanically-actuated mechanism coupled to the housing, the mechanically-actuated mechanism comprising a trigger that is actuatable to advance a medical device located in front of the elongated body along a surgical guide wire while the surgical guide wire concurrently extends through both the medical device and the elongated body; and
a nose portion that extends distally away from the elongated body and that retracts toward the elongated body against spring bias when the trigger is actuated to concurrently advance the elongated body and the medical device along the surgical guide wire while the medical device is obstructed by a surface in front of the medical device.

2. The hand-held medical device advancement apparatus of claim 1, wherein the surface comprises one or more of: a bone, a surgical plate.

3. The hand-held medical device advancement apparatus of claim 1, comprising a compression spring that provides the spring bias.

4. The hand-held medical device advancement apparatus of claim 3, wherein the compression spring has a maximum compression force amount below 150 N.

5. The hand-held medical device advancement apparatus of claim 1, wherein the nose portion is lockable in a locked configuration in which the nose portion remains immobile with respect to the elongated body.

6. The hand-held medical device advancement apparatus of claim 5, wherein the nose portion comprises one or more tabs that are extendable into one or more respective cavities in the elongated body to lock the nose portion in the locked configuration.

7. The hand-held medical device advancement apparatus of claim 1, comprising the medical device.

8. The hand-held medical device advancement apparatus of claim 1, wherein the mechanically-actuated mechanism further comprises a bar and a cam plate, the cam plate comprising an opening through which the surgical guide wire is extendable while also concurrently extending through the elongated body, the trigger coupled to the bar at a first end portion of the bar, the bar comprising a second end portion that engages the cam plate to move the cam plate based on actuation of the trigger.

9. The hand-held medical device advancement apparatus of claim 8, wherein the mechanically-actuated mechanism is configured, via actuation of the trigger, to move the cam plate to cinch the surgical guide wire as extending through the opening and to advance the hand-held medical device advancement apparatus along the surgical guide wire based on additional movement of the cam plate under control of the trigger.

10. An apparatus, comprising:
a housing defining a handle and a body intersecting the handle;
a mechanism coupled to the housing, the mechanism comprising an arm that is actuatable to advance a medical device located in front of the body along a surgical guide wire while the surgical guide wire concurrently extends through both the medical device and the body, the mechanism further comprising a bar and a plate, the plate comprising an opening through which the surgical guide wire is extendable while also concurrently extending through the body, the arm coupled to the bar at a first end portion of the bar, the bar comprising a second end portion that engages the plate to move the plate based on actuation of the arm.

11. The apparatus of claim 10, wherein the bar moves substantially parallel to a longitudinal axis of the body.

12. The apparatus of claim 10, comprising a nose portion that extends distally away from the body and that retracts toward the body against spring bias when the arm is actuated to concurrently advance the body and the medical device along the surgical guide wire while the medical device is obstructed by a surface in front of the medical device.

13. The apparatus of claim 12, wherein the nose portion is lockable in a locked configuration in which the nose portion remains immobile with respect to the body.

14. The apparatus of claim 13, wherein the apparatus comprises one or more tabs that are extendable into one or more respective cavities in the apparatus to lock the nose portion in the locked configuration.

15. The apparatus of claim 12, comprising:
a compression spring that provides the spring bias.

16. The apparatus of claim 15, wherein the compression spring has a maximum compression force amount below 150 N.

17. The apparatus of claim 10, wherein the mechanism is configured, via actuation of the arm, to move the plate to cinch the surgical guide wire and to move the apparatus along the surgical guide wire.

18. An apparatus, comprising:
a housing defining a handle and a body coupled to the handle; and
a nose portion extending distally away from the body, the nose portion being retractable toward the body against spring bias when the apparatus is actuated to concurrently move the body and a medical device in front of the nose portion along a surgical guide wire while the medical device is obstructed by a surface in front of the medical device.

19. The apparatus of claim 18, comprising:
an arm that is actuatable to move the body and the medical device forward along the surgical guide wire.

20. The apparatus of claim 18, wherein the nose portion is lockable in a locked configuration in which the nose portion remains retracted with respect to the body.
